(19) United States
(12) Patent Application Publication
(10) Pub. No.: US 2026/0179775 A1
(43) Pub. Date: Jun. 25, 2026

(54) DYNAMIC AI-POWERED SYSTEM FOR PERSONALIZED CLINICAL ASSESSMENT AND CARE MANAGEMENT

(71) Applicant: Ravi Hariprasad, MD, A PC, Folsom, CA (US)

(72) Inventor: Ravi Hariprasad, Folsom, CA (US)

(73) Assignee: Ravi Hariprasad, MD, A PC, Folsom, CA (US)

(21) Appl. No.: 19/541,570

(22) Filed: Feb. 17, 2026

Related U.S. Application Data
(63) Continuation of application No. PCT/US25/10240, filed on Jan. 3, 2025.
(60) Provisional application No. 63/617,813, filed on Jan. 5, 2024.

(51) Int. Cl. G16H 50/20

(52) U.S. Cl. CPC G16H 50/20

(57) ABSTRACT

Systems and methods for generating a patient-specific clinical assessment are disclosed. A processor receives patient health data corresponding to a patient. The patient health data is analyzed using a trained large language model (LLM) configured to process the patient health data. Clinical insights are determined based on the analysis of the patient health data using the trained LLM. A set of interactive prompts is generated for a patient interface based on the identified clinical insights. The set of interactive prompts may obtain additional information associated with the patient. A set of patient responses responsive to the generated set of interactive prompts are received. The clinical insights and the set of patient responses are displayed on a clinician interface.

### CROSS-REFERENCE TO RELATED APPLICATIONS

This application is a continuation of International Application No. PCT/US25/10240 filed January 3, 2025, which claims the priority benefit of U.S. Provisional Patent Application Ser. No. 63/617,813, filed January 5, 2024, each of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of healthcare information systems, and more specifically to the field of managing and processing healthcare data and individualizing patient care. Described herein are systems and methods for generating a patient-specific clinical assessment.

### BACKGROUND

According to the National Institute of Mental Health (NIMH), approximately one in five adults in the U.S. experience a mental illness in any given year. Common conditions include anxiety disorders, mood disorders (e.g., depression and bipolar disorder), substance use disorders, schizophrenia, and personality disorders. Mental health issues are also common among children and adolescents. The prevalence of mental health disorders in youth, such as anxiety, depression, and behavioral issues, has risen in recent years, with about 1 in 6 children ages 6-17 experiencing a mental health disorder each year.

Psychiatric care can take many forms, ranging from outpatient therapy to inpatient hospitalization. Various approaches can be used, for example, cognitive behavioral therapy (CBT), dialectical behavior therapy (DBT), psychodynamic therapy, medication, telehealth based therapy, etc. However, the viability of any therapy may be limited by the staff and their ability to gather and analyze information about the patient in need.

### SUMMARY

There is a need for new and useful system and method for generating a patient-specific clinical assessment. In particular, there is a need for systems, devices, and methods that can effectively analyse patient health data, including biometric information, historical medical records, and patient-reported symptoms, to generate personalized clinical insights.

A computer-implemented method for generating a patient-specific clinical assessment is disclosed. The method may include receiving, by a processor, health data corresponding to a patient. In some embodiments, the health data may include one or more of biometric data, historical medical data, and patient-reported symptoms. The method may further include analyzing, by the processor, the health data using a trained large language model (LLM) configured to process the health data. The method may further include identifying, by the processor, clinical insights based on the analysis of the health data using the trained LLM. In some embodiments, the clinical insights may include potential diagnoses, treatment recommendations, and prioritized patient conditions. The method may further include generating, by the processor, a set of interactive prompts for a patient interface based on the identified clinical insights, the set of interactive prompts being configured to obtain additional information associated with the patient. The method may further include receiving, by the processor, a set of patient responses responsive to the generated set of interactive prompts. The method may further include displaying, by the processor, the clinical insights and the set of patient responses on a clinician interface.

A computer-implemented system for generating a patient-specific clinical assessment is disclosed. The system includes a processor, and a memory communicably coupled to the processor, wherein the memory stores processor-executable instructions, which when executed by the processor, cause the processor to receive health data corresponding to a patient. In some embodiments, the health data may include one or more of biometric data, historical medical data, and patient-reported symptoms. The processor may further analyse the health data using a trained large language model (LLM) configured to process the health data. The processor may further identify clinical insights based on the analysis on the health data using the trained LLM. In some embodiments, the clinical insights may include potential diagnoses, treatment recommendations, and prioritized patient conditions. The processor may further generate a set of interactive prompts for a patient interface based on the identified clinical insights, the set of interactive prompts being configured to obtain additional information associated with the patient. The processor may further receive a set of patient responses responsive to the generated set of interactive prompts. The processor may further display the clinical insights and the set of patient responses on a clinician interface.

A non-transitory computer-readable storage medium for use in conjunction with a computing system is disclosed. The computer-readable storage medium being configured to store executable program instructions that, when executed by the computing system, cause the computing system to perform operations comprising receiving patient health data corresponding to a patient. In some embodiments, the patient health data may include one or more of biometric data, historical medical data, and patient-reported symptoms. The operations may further include analyzing the patient health data using a trained large language model (LLM) configured to process the patient health data. The operations may further include identifying clinical insights based on the analysis of the patient health data using the trained LLM. In some embodiments, the clinical insights may include potential diagnoses, treatment recommendations, and prioritized patient conditions. The operations may further include generating a set of interactive prompts for a patient interface based on the identified clinical insights. The set of interactive prompts being configured to obtain additional information associated with the patient. The operations may further include receiving a set of patient responses responsive to the generated set of interactive prompts. The operations may further include receiving a set of patient responses responsive to the generated set of interactive prompts.

### BRIEF DESCRIPTION OF THE DRAWINGS

There is a need for new and useful system and method for generating a patient-specific clinical assessment. In particular, there is a need for systems, devices, and methods that can effectively analyse patient health data, including biometric information, historical medical records, and patient-reported symptoms, to generate personalized clinical insights.

FIG. 1A illustrates a block diagram of an exemplary clinical assessment system for generating a patient-specific clinical assessment, in accordance with an embodiment of the present disclosure.

FIG. 1B illustrates a functional block diagram of the exemplary clinical assessment system, in accordance with an embodiment of the present disclosure.

FIG. 2 illustrates an exemplary flow diagram of processing patient health data, in accordance with an embodiment of the present disclosure.

FIG. 3 illustrates an exemplary flow diagram of training and initialization of the trained LLM, in accordance with an embodiment of the present disclosure.

FIG. 4 illustrates an exemplary flow diagram of dynamically updating the trained LLM, in accordance with an embodiment of the present disclosure.

FIG. 5 illustrates an exemplary flow diagram of semantic analysis and context-aware processing, in accordance with an embodiment of the present disclosure.

FIG. 6 illustrates an exemplary flow diagram of clinical workflow and decision support, in accordance with an embodiment of the present disclosure.

FIG. 7 illustrates an exemplary flow diagram of task allocation and workflow management, in accordance with an embodiment of the present disclosure.

FIG. 8 illustrates an exemplary dual-panel diagram depicting a patient interface and a clinician interface, in accordance with an embodiment of the present disclosure.

FIG. 9 illustrates an exemplary schematic diagram depicting encryption of the patient health data, in accordance with an embodiment of the present disclosure.

FIG. 10 illustrates an exemplary flow diagram of testing, deployment, and updates of the clinical assessment system, in accordance with an embodiment of the present disclosure.

FIG. 11 illustrates an exemplary architectural diagram of cloud-based data management, in accordance with an embodiment of the present disclosure.

FIG. 12 illustrates an exemplary flow diagram of real-time data handling within the clinical assessment system, in accordance with an embodiment of the present disclosure.

FIG. 13 illustrates an exemplary schematic diagram of the patient interface, in accordance with an embodiment of the present disclosure.

FIG. 14 illustrates an exemplary schematic diagram of the clinician interface, in accordance with an embodiment of the present disclosure.

FIG. 15 illustrates an exemplary flow diagram of data security and privacy protocol within the clinical assessment system, in accordance with an embodiment of the present disclosure.

FIG. 16 illustrates an exemplary flow diagram of interactive prompting and data capture process during patient intake, in accordance with an embodiment of the present disclosure.

FIG. 17 illustrates an exemplary flow diagram of analysing clinical data and generating treatment recommendations within the clinical assessment system, in accordance with an embodiment of the present disclosure.

FIG. 18 illustrates an exemplary flow diagram of communication flow and notification within the clinical assessment system, in accordance with an embodiment of the present disclosure.

FIG. 19 illustrates an exemplary architectural diagram depicting patient-centred care delivery, in accordance with an embodiment of the present disclosure.

FIG. 20 illustrates an exemplary data flow diagram of aggregation and processing of data from care activities, task performance, claims data, and patient outcomes into clinical insights via business intelligence dashboards, in accordance with an embodiment of the present disclosure.

FIG. 21 illustrates an exemplary flow diagram for the patient health data processing within the clinical assessment system, is illustrated, in accordance with an embodiment of the present disclosure.

FIG. 22 illustrates an exemplary data flow diagram depicting integration of various data streams into business intelligence dashboards for clinical insights, in accordance with an embodiment of the present disclosure.

FIG. 23 illustrates an exemplary workflow diagram for assessment data processing, showcasing the end-to-end handling of patient-submitted assessments to generate clinical insights, in accordance with an embodiment of the present disclosure.

FIG. 24 illustrates an exemplary workflow diagram for care plan development and monitoring, depicting the processes for creating, updating, and tracking patient-specific care plans dynamically, in accordance with an embodiment of the present disclosure.

FIG. 25 illustrates an exemplary workflow diagram for the seamless data flow into dashboards designed for real-time insights, in accordance with an embodiment of the present disclosure.

FIG. 26 illustrates an exemplary workflow for collecting, processing, and analyzing patient feedback, in accordance with an embodiment of the present disclosure.

FIG. 27 illustrates an exemplary use case scenario for the comprehensive management of a patient, in accordance with an exemplary embodiment of the present disclosure.

FIG. 28 illustrates an exemplary scenario, highlighting a care plan adjustment process triggered by monitoring patient progress and AI-based predictions, in accordance with an exemplary embodiment of the preset disclosure.

FIG. 29 illustrates a flowchart of a method for generating a patient-specific clinical assessment, in accordance with an embodiment of the present disclosure.

FIG. 30 illustrates a flowchart of a method for generating a patient-specific clinical assessment, in accordance with an embodiment of the present disclosure.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Interactions of patients with clinicians are severely time-bound as well as bound by the workload shouldered by the clinicians. Additionally, administrative works can act as a bar for interaction between patients and clinicians. Such limitations on clinicians may lead to acquisition of a partial history of the patients, resulting in treatment results that may be inadequate or inaccurate. Complexity, along with the volume of data, further increases when there is increased desire for personalized healthcare. Particularly, there are not enough specialized practitioners; it is one of the known challenges that the field of psychiatric care faces resulting in a lack of availability of critical services for patients in need and therefore a wide gap between the needs of patients and healthcare capabilities.

Conventional systems and techniques such as the Electronic Health Record (EHR) and other basic automation tools try to solve some of these problems, but do not offer a total solution. Generally, these systems and techniques have failed to utilize patient data to streamline diagnosis and treatment and optimize clinical workflow. Clinician burnout that is driven by administrative workload underscores the urgent necessity of a smart solution that can streamline tasks like data collection from patients, data entry, and data analysis. Disclosed herein are systems and methods for generating a patient-specific clinical assessment.

The systems and methods described herein solve a technical problem of efficiently processing and analyzing complex, heterogeneous patient data, including unstructured text, biometric information, and historical medical records, to generate patient-specific clinical assessments and insights. By utilizing a trained LLM within a cloud-based environment, the systems and methods address technical challenges such as real-time data normalization, semantic understanding of medical information, and secure integration with electronic medical records (EMR) systems. This approach reduces the computational overhead associated with traditional manual processes which improves the accuracy and timeliness of clinical recommendations and enables unified interoperability across disparate healthcare systems.

In particular, the technical problem sought to be solved by the present disclosure is to provide a system and method for generating patient-specific clinical assessments and insights that overcome limitations associated with manual data processing, fragmented healthcare data sources, and inefficiencies in clinical workflows. The present disclosure aims to enable the integration of heterogeneous patient data into a unified framework, utilizing advanced artificial intelligence (AI) techniques, including LLMs, to deliver accurate, real-time diagnostic support and personalized treatment recommendations while ensuring compliance with healthcare data security and interoperability standards.

In general, the systems described herein include an AI-augmented clinical intake assessment and care management system that provides a secure way to integrate artificial intelligence (AI) into existing medical care and management. The system functions to improve the efficacy and ease of clinical assessments, reduce practitioner burnout, and expand access to quality care. The system is engineered to enhance the efficiency and success of the healthcare delivery model by integrating LLM/AI technology with a cloud-based data management system. In some embodiments, the system represents a multiphase interaction model that may improve initial patient assessments, support longitudinal patient care, and optimize clinical documentation activities. The clinical intake assessment system may provide enhancements to the clinical intake processes in healthcare settings using an AI-augmented platform. The system may further provide actionable insight generation for clinicians through a real-time decision support interface to enhance diagnostic accuracy, provide personalized treatment plans, and reduce time burdens typically associated with patient data management. The system may further provide a patient-facing interface empowering patients to proactively participate in their care and a clinician-facing interface that streamlines workflow management and operational efficiency. The system may further provide context-aware processing capabilities that adapt over time to evolving patient data and medical knowledge.

The devices, methods, and/or methods of treatment (MOTs) described herein can be used to generate patient-specific clinical assessments, which provide a technical effect of enabling the integration of heterogeneous healthcare data sources, improving diagnostic accuracy, and streamlining clinical decision-making processes. This technical effect is achieved by utilizing a trained LLM to analyze structured and unstructured patient data, generate actionable clinical insights, and dynamically adapt patient and clinician interfaces for personalized healthcare delivery.

Some conventional systems and/or methods may utilize static rule-based algorithms or predefined templates for patient assessment and clinical data processing that fail to embrace the dynamic and adaptive needs of modern personalized healthcare. A potential drawback with such conventional solutions may include limited flexibility in adapting to heterogeneous data sources, inability to provide context-aware clinical insights, and a lack of real-time responsiveness to patient-specific changes or clinician inputs. Thus, the devices, methods, and/or MOTs described herein may provide an improvement over conventional solutions by employing a LLM trained to process and analyze diverse patient health data dynamically, utilizing advanced natural language processing techniques for data normalization, and incorporating retrieval-augmented generation (RAG) techniques for enhanced clinical recommendations. These improvements to the technical field of patient assessment and clinical data processing may enable personalized, adaptive, and contextually relevant patient care while reducing administrative burdens on clinicians and enhancing interoperability with existing healthcare infrastructure.

In general, the methods may include receiving, by a processor, patient health data corresponding to a patient. In some embodiments, the patient health data may include one or more of biometric data, historical medical data, and patient-reported symptoms. The method may further include analyzing, by the processor, the patient health data using a trained large language model (LLM) trained to process the patient health data. The method may further include identifying, by the processor, clinical insights based on the analysis of the patient health data using the trained LLM. In some embodiments, the clinical insights may include potential diagnoses, treatment recommendations, and prioritized patient conditions. The method may further include generating, by the processor, a set of interactive prompts for a patient interface based on the identified clinical insights, the set of interactive prompts being used to obtain additional information associated with the patient. The method may further include receiving, by the processor, a set of patient responses responsive to the generated set of interactive prompts. The method may further include displaying, by the processor, the clinical insights and the set of patient responses on a clinician interface.

The systems and devices described herein function to generate a patient-specific clinical assessment. In some embodiments, the systems and devices described herein function to generate patient-specific clinical insights. The systems and devices described herein may be used for healthcare, including but not limited to psychiatry, primary care, chronic disease management, and behavioural health but can additionally, or alternatively, be used for any suitable applications, clinical or otherwise, such as in research, education, or administrative settings. The systems and devices can be adapted to function for any suitable purpose, including processing patient data, providing decision support, automating clinical workflows, or generating personalized recommendations and insights based on user-provided or system-generated data.

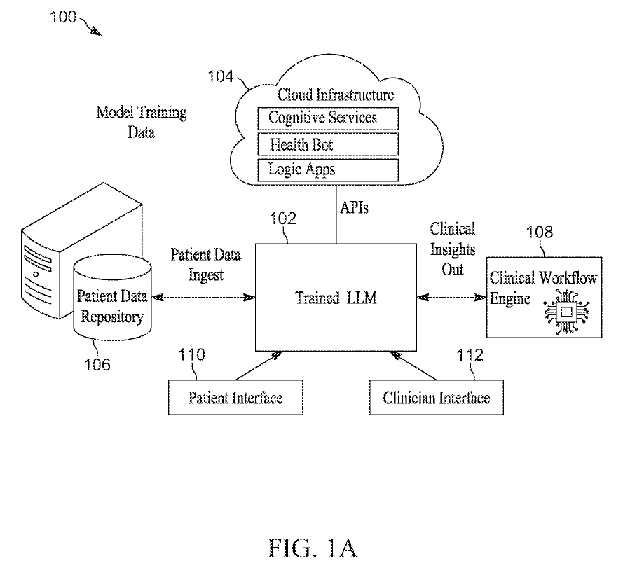

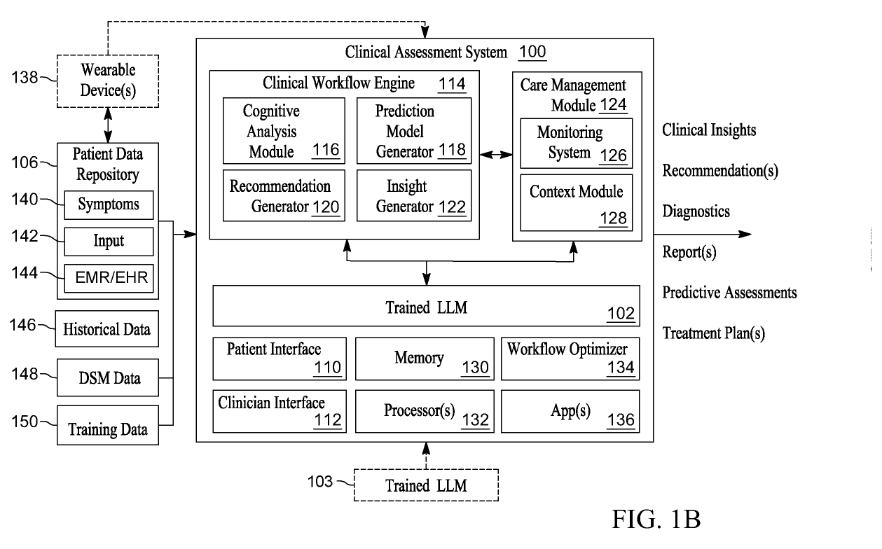

Referring now to FIG. 1A, a block diagram of an exemplary clinical assessment system 100 for generating a patient-specific clinical assessment, is illustrated, in accordance with an embodiment of the present disclosure. The clinical assessment system 100 is shown in FIG. 1A at a high level and may integrate advanced AI technologies, specifically a trained LLM 102, to analyze patient health data (e.g., patient data repository 106 or the like) and provide clinical insights (e.g., potential diagnoses, prioritized patient conditions, and treatment recommendations, etc.).

The clinical assessment system 100 may operate within a cloud-based infrastructure 104. The cloud-based infrastructure 104 may provide one or more services such as cognitive services, health bot, and logic apps, which collectively support AI processing, natural language processing, and workflow automation. The trained LLM 102 may serve as a central processing unit of the clinical assessment system 100 and may be trained on a comprehensive healthcare knowledge database (not shown) that may include, but is not limited to, psychiatric research, historical patient data, treatment outcomes, and medical literature. The trained LLM 102 may analyze received patient health data corresponding to a patient, which may include, but is not limited to patient biometric data (e.g., heart rate, blood pressure, oxygen saturation, etc.), patient historical medical data (e.g., previous diagnoses, prescribed medications, surgical history, etc.), and/or patient-reported symptoms (e.g., fatigue, chest pain, difficulty breathing, etc.). The patient health data may be received from, but is not limited to, a patient data repository 106 and may be processed through Application Programming Interfaces (APIs) connecting the patient data repository 106 to the trained LLM 102. The patient data repository 106 may include one or more input sources that may include, but are not limited to, electronic health records, wearable devices, and/or patient self-reports.

The trained LLM 102 may perform contextual processing of the patient health data to generate clinical insights, including potential diagnoses, prioritized patient conditions, and treatment recommendations. The generated clinical insights, which include potential diagnoses, prioritized patient conditions, and treatment recommendations, may be communicated to the clinical workflow engine 108 for further processing and operational integration. The clinical assessment system 100 may interface with one or more user components, for example a patient interface 110 and a clinician interface 112. The patient interface 110 enables the patient to complete structured intake forms, receive interactive health prompts and/or data, and engage interactively with the trained LLM 102 through dynamically generated prompts. The interactive prompts may be generated based on the clinical insights generated by the trained LLM 102 and may be contextualized to gather additional patient-specific information.

The clinician interface 112 may provide healthcare providers with real-time access to the clinical insights, the patient health data, and workflow management tools. Through the clinician interface 112, clinicians may review the LLM generated recommendations, validate diagnoses, and tailor treatment plans based on their professional judgement. The clinician interface 112 may also enable seamless synchronization of clinician-reviewed data with existing EMR systems.

The patient health data processing may include multiple stages, including data normalization and metadata augmentation. The clinical assessment system 100 may process the patient health data using natural language processing (NLP) techniques and map the patient health data to standardized medical coding systems (not shown) such as SNOMED CT or LOINC, for example. The processed and standardized data may be augmented with metadata, including temporal, locational, and categorical attributes, to support context-aware analysis by the trained LLM 102. The trained LLM 102 may employ RAG techniques by combining retrieval of particular documents with generative models to produce more accurate and contextually relevant output. This approach retrieves particular data from the healthcare knowledge database and may further include dynamically integrating the retrieved data into the clinical assessment process. The retrieved data may be selected based at least in part on the patient health data, prompts, insights, and/or other input or analysis performed by system 100. The trained LLM 102 may further perform continuous learning and adaptation processes.

Referring now to FIG. 1B, a functional block diagram of the exemplary clinical assessment system 100 is illustrated, in accordance with an embodiment of the present disclosure. The clinical assessment system 100 integrates various hardware and software components to enable efficient data processing. The clinical assessment system 100 may include, but is not limited to, the trained LLM 102 (and/or external trained LLM 103), the clinical workflow engine 108, the patient interface 110, the clinician interface 112, a care management module 124, a memory 130, one or more processors 132, a workflow optimizer 134, and one or more applications 136.

The trained LLM 102 is an analytical component of the clinical assessment system 100. Both the trained LLM 102 and the external trained LLM 103 may operate as an advanced LLM or other machine learning framework. The trained LLM 102 (and/or external trained LLM 103) may be implemented using frameworks such as TensorFlow, PyTorch, or other machine learning platforms, and may operate locally or in a cloud-based environment. Alternate embodiments may include multiple AI/ML models to handle specialized tasks, such as predictive modelling or natural language processing. In some embodiments, the trained LLM 102 (and/or external trained LLM 103) may be trained on training data 150 received from the healthcare knowledge database (not shown). The training data 150 includes, but is not limited to, psychiatric research, historical patient data, treatment outcomes, and medical literature to ensure that the trained LLM 102 (and/or the external trained LLM 103) are versed in both theoretical and practical medical knowledge. To achieve this, the trained LLM 102 (and/or external trained LLM 103) employs RAG techniques, which allow it to dynamically retrieve relevant data from connected repositories such as the historical data repositories 146 and Diagnostic and Statistical Manual of Mental Disorders (DSM) data sources 148. The retrieved information is then combined with the patient data to generate insights that are both comprehensive and individualized.

The clinical workflow engine 108 may include one or more modules or a plurality of modules, including a cognitive analysis module 116, a prediction model generator 118, a recommendation generator 120, and an insight generator 122. These modules collectively manage the processing of the patient health data. In some embodiments, the clinical workflow engine 108 may include additional modules for advanced analytics or be integrated with external systems for multi-department coordination.

The cognitive analysis module 116 provide or utilize a service for integrating advanced text analytics and semantic data processing to enhance the performance of the trained LLM 102. In some embodiments, the cognitive analysis module 116 may provide or utilize a service to streamline medical transcription processes and data capturing. In some embodiments, the cognitive analysis module 116 may provide or utilize a service to leverage available medical and patient knowledge bases to reinforce real-time patient data analyses executed by the trained LLM 102 (and/or external trained LLM 103).

The prediction model generator 118 may utilize inputs, feedback, and clinical insights to generate predictive models to enhance the precision of health assessments and/or treatments. Such models may be used to predict exacerbations in chronic conditions before such conditions manifest overtly. In some embodiments, the prediction model generator 118 may generate models that predict potential disruptions or slowdowns and system 100 may generate automated workflows to address such disruptions or slowdowns proactively. In some embodiments, the prediction model generator 118 may generate models that provide or refine healthcare analytics to improve decision-making for the clinician and/or patient. The care management module 124 includes a monitoring system 126 and a context module 128 to support the real-time tracking of patient progress and the contextualization of data. These sub-modules work in tandem with the clinical workflow engine 108 to ensure personalized and adaptive patient care. Alternate embodiments of the care management module 124 may incorporate predictive monitoring capabilities or AI-driven alerts for high-risk scenarios.

The patient interface 110 is a patient-centric platform designed to interact directly with patients. The patient interface 110 allows for the collection of patient-reported symptoms, displays clinical insights, and dynamically adapts interactive prompts based on analysis of the trained LLM 102 (and/or external trained LLM 103). The patient interface 110 may be implemented as a web-based application, a mobile app, or integrated with the wearable devices 138.

The clinician interface 112 is tailored for healthcare providers or clinicians to offer access to patient health data, LLM generated insights, and workflow management tools. The clinician interface 112 enables clinicians to review, validate, and update care plans in real time. The clinician interface 112 may support integration with an EMR and/or an electronic health record (i.e., EMR/EHR 144) and may include customization options for individual provider workflows. In general, EMR/EHR 144 may include an electronic medical record, an electronic health record, or both.

The applications 136 within the clinical assessment system 100 provide supplementary functionalities, such as task automation, data visualization, and remote access to the clinical assessment system 100. These applications 136 can operate on various hardware platforms, including computing devices, desktops, tablets, and mobile devices.

The processors 132 may include one or more processors, such as central processing units (CPUs), graphics processing units (GPUs), or specialized accelerators designed for machine learning tasks. The one or more processors may include one or more devices capable of executing instructions stored by the memory 130, to perform operations and/or communications amongst systems, engines, modules, and/or devices described herein. The memory 130 may include one or more non-transitory computer-readable storage media, such as solid-state drives (SSDs), dynamic random-access memory (DRAM), or flash storage devices. The memory 130 may store instructions and data that are usable in combination with processors 132 to execute the processes and/or algorithms described herein as well as to execute or interface with trained LLM 102. The memory 130 may also function to store or have access to the trained LLM 102.

Input sources may include wearable devices 138 and the patient data repository 106, which may include patient-reported symptoms 140, input 142, and the EMR/EHR 144. The input sources may also include historical medical data 146, and DSM data 148. These input sources supply real-time or historical data, which is processed by the clinical assessment system 100 for patient analysis. The clinical assessment system 100 may also include training datasets 150 for continuous refinement of the trained LLM 102 (and/or external trained LLM 103).

By way of an example, the patient interface 110, the wearable devices 138, and/or other input sources may provide patient health data (or other data) corresponding to a patient, to the clinical workflow engine 108 for further processing. In some embodiments, the patient health data (or other data) may include, but is not limited to, one or more of biometric data from the wearable devices 138, historical medical data 146, and patient-reported symptoms 140 or other patient indicated input. The clinical workflow engine 108 may receive the patient health data. The cognitive analysis module 116 of the clinical workflow engine 108 may normalize the patient health data using natural language processing techniques and data standardization techniques. The context module 128 within the care management module 124 may further augment the normalized data with metadata corresponding to the patient health data. In some embodiments, the metadata may be received from one or more of the input sources, such as the wearable devices 138, electronic health records from the EMR/EHR 144, or patient self-reported symptoms 140, etc.. The metadata may include temporal attributes, locational attributes, and/or categorical attributes corresponding to the patient health data. The cognitive analysis module 116 may further map the normalized and/or augmented data to a standardized medical coding system, such as SNOMED CT or LOINC. The workflow engine 114 may encrypt the patient health data using an end-to-end encryption protocol to ensure data privacy and security.

The trained LLM 102, as part of the clinical workflow engine 108, analyzes the patient health data to generate clinical insights. The insight generator 122 performs this analysis by using the contextual processing of the trained LLM 102, which personalizes the identified clinical insights. The contextual processing may be based, at least in part, on historical medical data 146, real-time updates from the wearable devices 138 associated with the patient, and diagnostic criteria from the DSM data 148. The insight generator 122 using the trained LLM 102 identifies clinical insights, which may include, but are not limited to, potential diagnoses, treatment recommendations, and prioritized patient conditions, or the like. The monitoring system 126 of the care management module 124 may assist in dynamically tracking symptom progression and/or health trends to further refine the identified clinical insights. The recommendation generator 120 may generate a set of interactive prompts for the patient interface 110 based on the identified clinical insights. These interactive prompts may be used to obtain additional information associated with the patient. The recommendation generator 120, in conjunction with the trained LLM 102, may dynamically adapt the set of prompts using a decision-tree algorithm based on the patient responses. The patient interface 110 may receive and transmit these responses back to the clinical workflow engine 108, where the insight generator 122 can process the inputs 142 to refine clinical insights. The updated clinical insights and patient responses may be displayed on the clinician interface 112, allowing healthcare providers to validate or modify the care recommendations.

The processors 132 may further generate a set of interactive prompts for a patient interface 110 based on the identified clinical insights. The set of interactive prompts may be provided in the patient interface 110 and/or clinician interface 112 to cause processor 132 to obtain additional information associated with the patient. The processor 132 may further dynamically adapt the set of prompts based on the set of patient responses, using a decision-tree algorithm implemented by the trained LLM 102. The processor 132 may further receive a set of patient responses responsive to the generated set of interactive prompts. The processor 132 may further display the clinical insights and the set of patient responses on the clinician interface 112.

The trained LLM 102 (and/or external trained LLM 103) may be dynamically updated with the patient health data and clinician feedback using the workflow engine 114 to improve diagnostic accuracy and treatment recommendations over time. The clinical workflow engine 108 may synchronize the identified clinical insights and clinician-reviewed data with the EMR/EHR 144 to maintain up-to-date patient records.

In another example, consider a patient, Sarah, a 45-year-old with a history of Type 2 diabetes, generalized anxiety disorder (GAD), and mild hypertension. Sarah uses the wearable device 138 to track her physical activity, glucose levels, and heart rate. Additionally, she provides patient-reported symptoms 140 such as fatigue and occasional dizziness through the patient interface 110. This example demonstrates how the clinical assessment system 100 processes her health data (i.e., patient health data) to generate personalized clinical insights and care recommendations.

Sarah’s wearable device 138 transmits real-time biometric data, including her glucose levels, heart rate variability, and daily step count, to the clinical workflow engine 108. In parallel, Sarah logs her fatigue severity and dietary intake (i.e., input 142) using the patient interface 110, while her historical medical data 146, such as past treatments and lab results, is retrieved from the EMR/EHR 144. Additionally, the clinical assessment system 100 incorporates DSM data 148 to cross-reference diagnostic criteria for her anxiety symptoms.

The cognitive analysis module 116 within the clinical workflow engine 108 normalizes this patient health data using natural language processing (NLP) and data standardization techniques. Metadata such as the time of day, location, and context of patient-logged (i.e., Sarah) symptoms are appended by the context module 128 in the care management module 124 to ensure that the data is enriched with temporal, locational, and/or categorical attributes. The normalized and augmented data is then mapped to a standardized medical coding system, such as SNOMED CT, for interoperability.

The trained LLM 102, integrated with the clinical workflow engine 108, analyzes Sarah’s health data (i.e., patient health data) to identify clinical insights. The insight generator 122 processes this patient health data using contextual processing techniques, utilizing her historical medical records and real-time updates from her wearable device 138. For Sarah, the insight generator 122 identifies a potential diagnosis of prediabetic neuropathy based on her elevated glucose levels and reported symptoms of fatigue and dizziness. The insight generator 122 also identifies a recommendation for cognitive behavioral therapy (CBT) to manage her anxiety, tailored to DSM data 148. The insight generator 122 also identifies a prioritized condition list with her fluctuating glucose levels flagged as urgent for immediate intervention. The monitoring system 126 tracks Sarah’s symptom progression, dynamically updating the clinical insights to reflect trends in her glucose levels and heart rate.

The recommendation generator 120 generates interactive prompts for the patient interface 110 based on the generated clinical insights. Sarah is asked to answer the interactive prompts about her dietary habits, stress levels, and sleep quality. The trained LLM 102, in conjunction with the recommendation generator 120, dynamically adapts these interactive prompts using a decision-tree algorithm to ensure that the questions are personalized and relevant. For example, if Sarah indicates high stress levels, the clinical assessment system 100 generates additional prompts about recent life changes or work-related stressors. Sarah’s responses are transmitted back to the clinical workflow engine 108, where the insight generator 122 refines its recommendations based on her inputs. The refined clinical insights and Sarah’s responses are displayed on the clinician interface 112. The provider sees a flagged alert for immediate glucose level management. The provider also sees a recommendation to adjust Sarah’s dietary plan and increase her physical activity. The provider also sees a proposed referral to a therapist for CBT sessions. The clinician interface 112 provides an interactive dashboard that allows the provider to modify care plans in real time and synchronize the updates with Sarah’s EMR/EHR 144.

The clinician assessment system 100 dynamically updates the trained LLM 102 with Sarah’s new data and the provider’s feedback, improving the accuracy of future insights. Using a RAG technique, the cognitive analysis module 116 retrieves the latest clinical research on prediabetic neuropathy and anxiety management from the healthcare knowledge database. The clinical assessment system 100 generates and delivers the clinical insights such as identified conditions, including potential prediabetic neuropathy and high-stress levels, prioritized for intervention. The clinical assessment system 100 also generates and delivers treatment recommendations such as tailored dietary adjustments, physical activity plans, and CBT sessions. The clinical assessment system 100 also generates and delivers interactive reports such as a summary of Sarah’s glucose trends, anxiety triggers, and real-time symptom progression for clinician review. The clinical assessment system 100 also generates and delivers predictive assessments such as a projection of Sarah’s glucose trends based on her current dietary patterns and physical activity levels. The clinical assessment system 100 also generates and delivers personalized treatment plans such as updated care recommendations synchronized with Sarah’s EMR/EHR 144 for continuity of care.

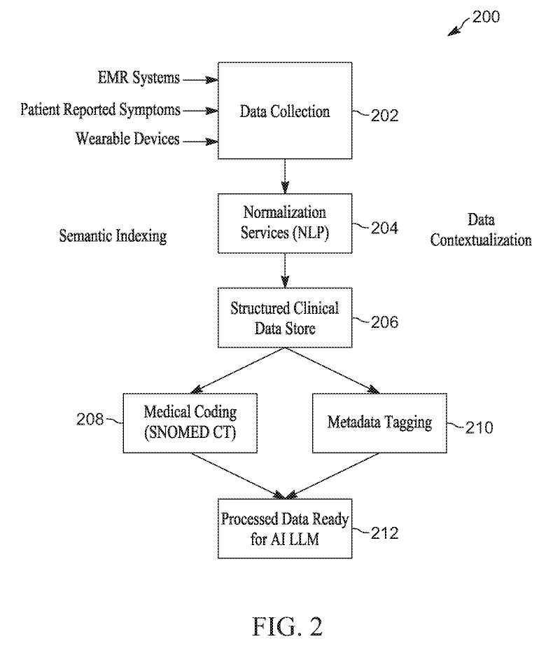

Referring now to FIG. 2, an exemplary flow diagram 200 of processing the patient health data, is illustrated, in accordance with an embodiment of the present disclosure. In some embodiments, the flow diagram 200 may be carried out by one or more processors/components of the clinical assessment system 100. In some embodiments, the flow diagram 200 may be carried out by one or more processors external to system 100. The flow diagram 200 demonstrates the operations involved in processing patient health data for subsequent analysis by the trained LLM 102.

The process begins at a data collection block 202, which receives patient health data from the input sources. These input sources may include the wearable devices 138 and/or the patient data repository 106, which may include patient-reported symptoms 140, input 142, and the EMR/EHR 144, for example. The patient health data received may include, but is not limited to, one or more of biometric data from the wearable devices 138, historical medical data 146, and patient-reported symptoms 140.

The received data is subsequently passed to a normalization services block 204, which employs NLP techniques to standardize the patient health data. This block 204 involves semantic indexing to ensure that the patient health data from the input sources is translated into a uniform format. The normalization process resolves inconsistencies in terminology, structure, and representation of the patient health data. For example, NLP may standardize patient-reported symptoms or wearable device metrics into a structured format compatible with the clinical assessment system 100. The normalized data is stored within a structured clinical data store 206, which acts as a central repository for organized and indexed patient health data. This structured format ensures that the patient health data is readily accessible for subsequent processing tasks. The structured clinical data store 206 enables data contextualization. The contextualized data from the structured clinical data store 206 is further processed through two parallel pathways: medical coding block 208 and metadata tagging block 210. The medical coding block 208 maps the structured data to standardized medical coding systems such as SNOMED CT or LOINC. This ensures interoperability across various healthcare systems and platforms, allowing consistent interpretation of clinical data. For instance, symptoms and diagnoses are encoded in a standardized format, which can be universally understood.

Concurrently (or subsequently), the metadata tagging block 210 augments the patient health data with metadata. These metadata may include temporal data (e.g., the timing of symptom onset), locational data (e.g., where the patient received care), and categorical data (e.g., type of medical intervention). For example, metadata may indicate a correlation between specific patient-reported symptoms and time of day. Both the coded data from the medical coding block 208 and the patient health data from the metadata tagging module 210 are combined to form a processed data set 212, which is ready for analysis by the trained LLM 102. The processed data 212 serves as an input to the trained LLM 102.

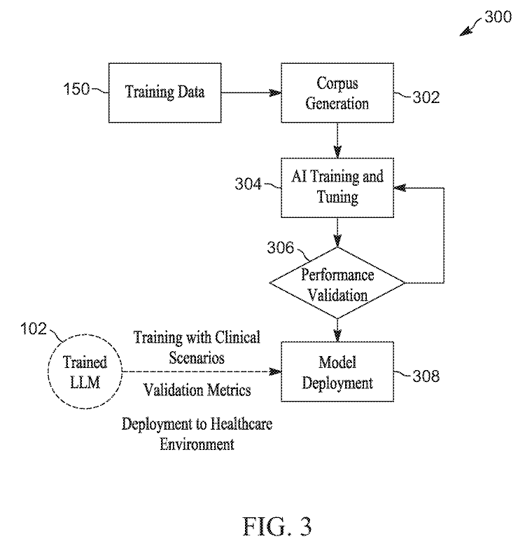

Referring now to FIG. 3, an exemplary flow diagram 300 of training and initialization of the trained LLM 102, is illustrated, in accordance with an embodiment of the present disclosure. In some embodiments, the flow diagram 300 may be carried out by one or more processors/components of the clinical assessment system 100. In some embodiments, the flow diagram 300 may be carried out by one or more processors external to system 100. The flow diagram 300 represents the structured approach to building, training, validating, and deploying the trained LLM 102 to operate as an analytical component of the clinical assessment system 100. The process begins with the training data 150, which forms the foundational knowledge base for training the LLM 102. The training data 150 may include, but is not limited to, a diverse range of healthcare-related data such as psychiatric research, historical patient data, treatment outcomes, and/or medical literature.

The training data 150 may be fed into the corpus generation module 302, which preprocesses and structures the training data 150 into a training-ready format. This module 302 performs tasks such as data cleaning, tokenization, and semantic tagging. The corpus generation module 302 ensures that the raw training data 150 is transformed into an optimized, structured corpus that captures the semantic and contextual degrees utilized for effective LLM training. The structured training data is then passed to an AI training and tuning module 304, where the initial training of the LLM 102 occurs. This module 304 utilizes machine learning frameworks such as TensorFlow or PyTorch to train the LLM 102 on the training data 150. The training process involves adjusting the LLM 102 parameters through iterative learning cycles to optimize performance.

Once the initial training is complete, the trained LLM 102 undergoes performance validation 306, which serves as an evaluation step. The performance validation module 306 assesses the trained LLM 102 against predefined validation metrics, including accuracy, recall, precision, and contextual understanding. This step may also include testing the trained LLM 102 with real-world clinical scenarios to gauge its effectiveness in generating clinical insights, diagnoses, and recommendations. If the trained LLM 102 fails to meet the performance thresholds, the training and tuning module 304 may be re-engaged for additional refinement.

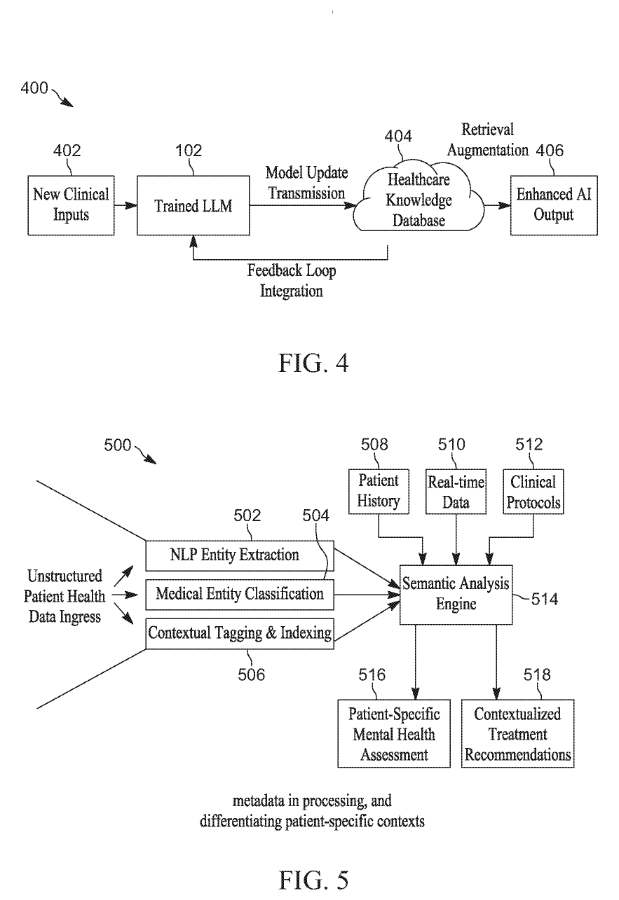

Following successful validation, the trained LLM 102 progresses to model deployment 308. At this stage, the trained LLM 102 is prepared for integration into the clinical assessment system 100. The deployment process involves embedding the trained LLM 102 into system architecture of the clinical assessment system 100, including integration with the clinical workflow engine 108, the patient interface 110, and the clinician interface 112. The model deployment block 308 ensures seamless operation of the trained LLM 102 in a live healthcare environment. The trained LLM 102 may be initialized within the healthcare environment. The trained LLM 102 undergoes additional training with clinical scenarios provided by real-world healthcare settings, as will be described in greater detail in FIG. 4.

Referring now to FIG. 4, an exemplary flow diagram 400 of dynamically updating the trained LLM 102, is illustrated, in accordance with an embodiment of the present disclosure. In some embodiments, the flow diagram 400 may be carried out by one or more processors/components of the clinical assessment system 100. In some embodiments, the flow diagram 400 may be carried out by one or more processors external to system 100. The flow diagram 400 depicts the iterative process by which the trained LLM 102 is dynamically updated with the patient health data.

New clinical inputs 402, which may include the patient health data, real-time updates from the wearable devices 138, the EMR/EHR 144, and clinician feedback via the workflow engine 114 may be received. These new clinical inputs 402 provide real-time patient health data for continuous refinement of the trained LLM 102. The new clinical inputs 402 are processed by a trained LLM 102, which serves as a component for analyzing and integrating the new clinical inputs 402. The trained LLM 102 utilizes advanced machine learning techniques, including contextual analysis, semantic understanding, and pattern recognition, to extract meaningful insights from the new clinical inputs 402. The trained LLM 102 also incorporates existing metadata and standardized coding systems (e.g., SNOMED CT, LOINC) to ensure interoperability and consistency in the analysis. The processed data are transmitted as a model update transmission to the healthcare knowledge database 404. The healthcare knowledge database 404 functions as a centralized repository of accumulated clinical knowledge, including prior training datasets, medical literature, and historical patient data. This healthcare knowledge database 404 is continuously updated through RAG techniques to enable the LLM to expand its contextual and semantic understanding dynamically.

The RAG techniques employed by the healthcare knowledge database 404 retrieves relevant data subsets from the healthcare knowledge database 404 to supplement the processing capabilities of the trained LLM 102. By doing so, the trained LLM 102 utilizes both historical knowledge and real-time updates (e.g., new clinical research studies, recent diagnostic guidelines, updated medication protocols, or real-time wearable device data) to enhance its predictive accuracy and contextual relevance. Based on the enriched knowledge from the healthcare knowledge database 404, the trained LLM 102 generates enhanced AI outputs 406. These outputs may include potential diagnoses, treatment recommendations, prioritized clinical conditions, predictive health assessments, and adaptive care plans tailored to individual patient needs. The enhanced AI outputs 406 are further validated and contextualized through a feedback loop integration. The feedback loop integration enables continuous improvement of the trained LLM 102. Feedback may be received from clinicians using the clinician interface 112 and/or patient responses using the patient interface 110.

Referring now to FIG. 5, an exemplary flow diagram 500 of semantic analysis and context-aware processing is illustrated, in accordance with an embodiment of the present disclosure. FIG. 5 depicts the process by which unstructured clinical data is transformed into patient-specific mental health assessments and contextualized treatment recommendations through a series of semantic and contextual analysis steps. In some embodiments, the flow diagram5300 may be carried out by one or more processors/components of the clinical assessment system 100. In some embodiments, the flow diagram 500 may be carried out by one or more processors external to system 100.

Unstructured patient health data is input into the system. In some embodiments, the patient health data may include, but is not limited to, one or more of biometric data from the wearable devices 138, historical medical data 146, patient-reported symptoms 140, and the EMR/EHR 144. This unstructured patient health data is processed through multiple analytical stages to extract relevant medical information and provide clinical insights. The process may include NLP entity extraction 502, which utilizes NLP techniques to identify key medical entities, such as symptoms, conditions, medications, and lab results, from the unstructured clinical data. The extracted entities serve as elements for subsequent analyses.

The extracted entities are then categorized through medical entity classification 504, where the clinical assessment system 100 assigns standardized medical codes, such as SNOMED CT or LOINC, to the identified entities. This classification ensures interoperability and enables consistent interpretation across healthcare systems. In parallel, the clinical assessment system 100 performs contextual tagging and indexing 506 to enrich the patient health data with metadata. These metadata include, but are not limited to, temporal information (e.g., event timestamps), locational details (e.g., healthcare facility), and/or categorical classifications (e.g., patient demographics).

The outputs from the NLP entity extraction 502, the medical entity classification 504, and the contextual tagging and indexing 506 are transmitted to the semantic analysis engine 514, which integrates these components with additional data sources, including patient history 508, real-time data 510, and clinical protocols 512. The patient history 508 may include longitudinal medical records, such as past diagnoses and treatments. The real-time data 510 includes dynamic inputs, such as wearable device readings and recent lab results. The clinical protocols 512 encompass evidence-based guidelines and best practices.

The semantic analysis engine 514 utilizes machine learning models, including the trained LLM 102, to perform advanced semantic and contextual processing. By synthesizing data from multiple sources, the semantic analysis engine 514 may generate one or more primary outputs, for example patient-specific mental health assessments 516 and contextualized treatment recommendations 518. The patient-specific mental health assessments 516 provide a detailed understanding of current mental health status of the patient, including prioritized conditions, potential risk factors, and symptom trajectories. These assessments are tailored to the individual clinical context. The contextualized treatment recommendations 518 offer clinical insights for healthcare providers, such as personalized treatment plans, medication adjustments, and lifestyle intervention strategies. These recommendations are aligned with the patient’s unique clinical profile and adhere to established medical guidelines.

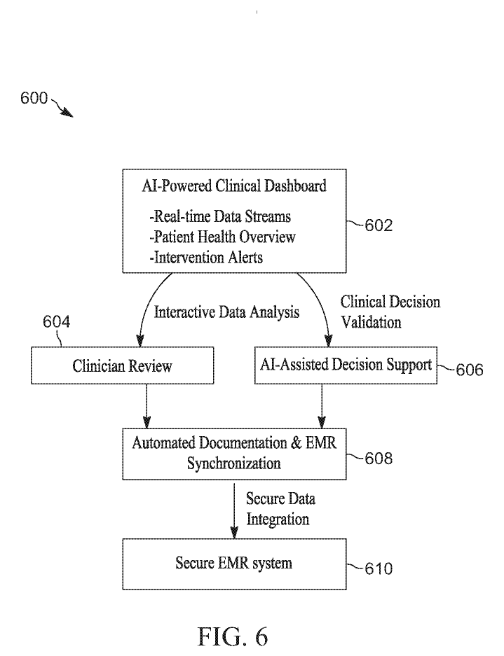

Referring now to FIG. 6, an exemplary flow diagram 600 of clinical workflow and decision support is illustrated, in accordance with an embodiment of the present disclosure. In some embodiments, the flow diagram 600 may be carried out by one or more processors/components of the clinical assessment system 100. In some embodiments, the flow diagram 600 may be carried out by one or more processors external to system 100. FIG. 6 depicts the interconnected components and processes involved in enabling real-time clinical decision-making and seamless integration with EMR/EHR 144.

The clinical dashboard 602 may be integrated in the clinician interface 112, which provides clinicians with a consolidated view of real-time data streams, a comprehensive patient health overview, and intervention alerts. This clinical dashboard 602 acts as the primary interface for interacting with the clinical insights and serves as a decision-making hub. The clinician dashboard 602 provides clinicians with clinical insights in a user-friendly format. The clinical dashboard 602 enables interactive data analysis and clinical decision validation through one or more pathways: clinician review 604 and AI-assisted decision support 606. In the clinician review pathway 604, healthcare providers manually evaluate the clinical insights, utilizing their expertise to validate or modify the recommendations. This clinician review pathway 604 ensures that clinical decisions align with established medical practices and patient-specific contexts.

Alternatively, the AI-assisted decision support pathway 606 utilizes advanced algorithms within the clinical workflow engine to autonomously suggest potential treatment plans, identify critical risk factors, and flag inconsistencies in the patient health data. This pathway streamlines the decision-making process, thereby allowing clinicians to focus on high-priority cases and improving efficiency in high-volume clinical settings. Once decisions are validated or refined through either pathway, the flow diagram 600 proceeds to automated documentation and EMR synchronization indicated in block 608. This block 608 involves the automatic generation of clinical notes, treatment plans, and diagnostic summaries based on the finalized decisions. The documentation is formatted to comply with standards such as FHIR (Fast Healthcare Interoperability Resources) to ensure compatibility with diverse EMR systems. The documentation generated at block 608 is securely integrated with the secure EMR system 610 through a robust data integration framework. This framework employs encryption protocols and role-based access controls to maintain the confidentiality and integrity of patient health data.

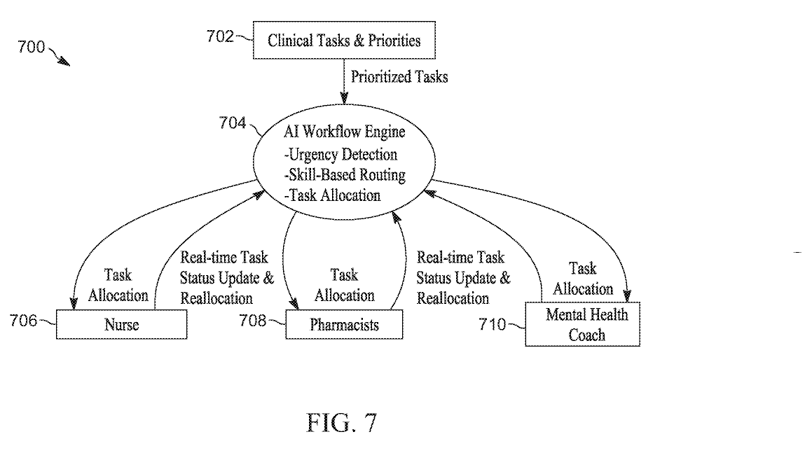

Referring now to FIG. 7, an exemplary flow diagram 700 of task allocation and workflow management is illustrated, in accordance with an embodiment of the present disclosure. In some embodiments, the flow diagram 700 may be carried out by one or more processors/components of the clinical assessment system 100. In some embodiments, the flow diagram 700 may be carried out by one or more processors external to system 100. FIG. 7 depicts operational framework of a task allocation system integrated with an AI workflow engine 704 to ensure optimized resource utilization and efficient management of clinical workflows. The clinical tasks and priorities module 702 receives a list of tasks based on current clinical demands, patient care priorities, and organizational objectives. This module 702 organizes the tasks and assigns priority levels, ensuring that high-urgency tasks are flagged for immediate attention. For example, tasks such as medication review or patient monitoring with critical conditions may be prioritized over routine follow-ups.

The prioritized tasks are transmitted to the AI workflow engine 704, which is equipped with capabilities for urgency detection, skill-based routing, and task allocation. The urgency detection component evaluates the criticality of each task using real-time patient data and predefined clinical protocols. The skill-based routing functionality maps tasks to appropriate healthcare providers based on their expertise, availability, and workload. The task allocation mechanism ensures that each task is dynamically assigned to the most suitable team member. The task allocation process is illustrated through three representative roles in FIG. 7, nurse 706, pharmacist 708, and mental health coach 710. The AI workflow engine 704 allocates tasks to these roles based on specific criteria. For instance, medication reconciliation tasks may be routed to the pharmacist 708, while patient counselling activities could be allocated to the mental health coach 710. Similarly, tasks such as vital sign monitoring may be assigned to the nurse 706.

Once tasks are assigned, the clinical assessment system 100 enables real-time task status updates and reallocation. This feature ensures continuous monitoring of task progress and allows the AI workflow engine 704 to dynamically reallocate tasks in response to delays, resource availability changes, or unforeseen circumstances. For example, if the nurse 706 encounters an unexpected workload, the system may reassign non-critical tasks to other team members, such as the mental health coach 710.

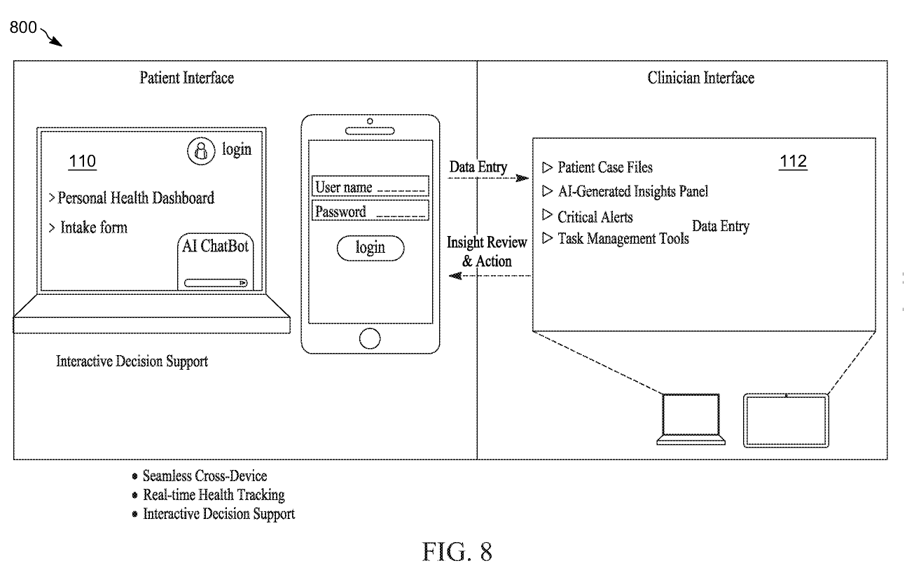

Referring now to FIG. 8, an exemplary dual-panel diagram 800 depicting the patient interface 110 and the clinician interface 112, is illustrated, in accordance with an embodiment of the present disclosure. The left side of the dual-panel diagram 800 represents the patient interface 110, which is designed for direct interaction with patients to enable data input, real-time health tracking, and interactive decision support. The patient interface 110 can be accessed through multiple devices, including desktop computers, mobile phones, and tablets. The patient interface 110 provides several functionalities such as a Personal Health Dashboard which enables patients to view their health metrics, treatment progress, and personalized insights generated by the trained LLM 102. The patient interface 110 further provides an intake form in which patients can input symptoms, medical history, and lifestyle information, which is processed and analyzed by the cognitive analysis module 116 of the clinical workflow engine 108. The patient interface 110 further provides an AI Chatbot which is embedded within the interface 110, the AI chatbot provides a conversational interface for patients to ask questions, receive guidance, and clarify medical instructions.

The right side of the dual-panel diagram 800 represents the clinician interface 112, which provides healthcare providers with tools for reviewing and managing patient health data, as well as LLM generated clinical insights. The clinician interface 112 offers features such as patient case files in which clinicians can access the patient health data, including the historical medical data 146, real-time updates from wearable devices 138, and the EMR/EHR 144. The clinician interface 112 further offers an AI-generated insights panel which displays clinical insights, such as potential diagnoses, prioritized conditions, and treatment recommendations, generated by the insight generator 122. The clinician interface 112 further offers critical alerts provided for urgent matters, such as potential medication contraindications or significant health deterioration. The clinician interface 112 further offers task management tools which enable workflow management by allowing clinicians to assign tasks, track progress, and collaborate with other healthcare providers.

Data flow between the patient interface 110 and the clinician interface 112 is bi-directional. Patients enter data using intake forms and/or the AI chatbot, which is processed by the clinical workflow engine 108. The resulting clinical insights and updates are transmitted to the clinician interface 112 for review and validation. Conversely, clinicians can update care plans or recommendations, which are communicated back to the patient interface 110 for patient action or acknowledgment.

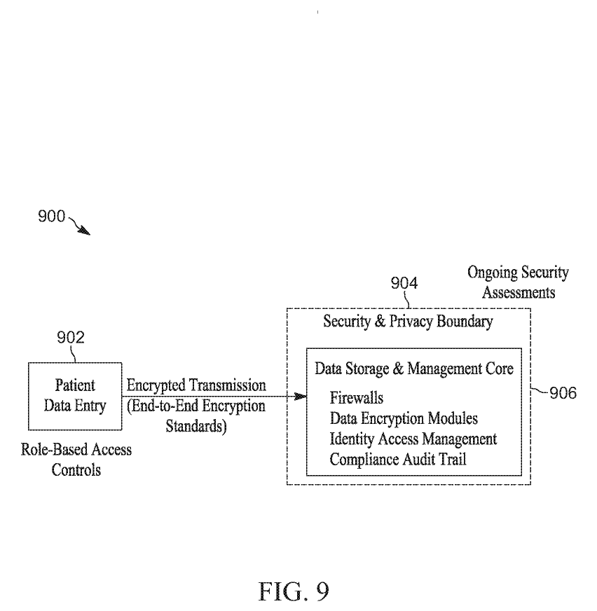

Referring now to FIG. 9, an exemplary schematic diagram 900 depicting encryption of the patient health data, is disclosed, in accordance with an embodiment of the present disclosure. The schematic diagram 900 depicts the secure handling, storage, and management of the patient health data within the clinical assessment system 100. The process begins with the patient data entry module 902, which represents the point at which patient health data, such as one or more of biometric data from the wearable devices 138, historical medical data 146, and patient-reported symptoms 140, is entered into the clinical assessment system 100. This patient health data may be entered via the patient interface 110 or other integrated data collection devices, such as the wearable devices 138 or the EMR/EHR 144. To ensure role-based access control, the clinical assessment system 100 enforces strict authentication and authorization protocols, thereby preventing unauthorized access to sensitive information. Data entered through the data entry module 902 is transmitted securely using end-to-end encryption standards, thereby ensuring that data integrity and confidentiality are maintained during transmission.

The encrypted data is then directed into the Security and Privacy Boundary 904, which defines the protected perimeter of data storage and management infrastructure of the clinical assessment system 100. The boundary 904 employs multiple layers of security controls to ensure robust protection against unauthorized access or breaches. Components within the boundary 904 may include firewalls, data encryption modules, identity access management (IAM), and compliance audit trail. In some embodiments, the firewalls filter incoming and outgoing network traffic, blocking unauthorized access and preventing potential threats. In some embodiments, the data encryption modules ensure that data remains encrypted both in transit and at rest, utilizing encryption protocols that comply with healthcare standards such as HIPAA. In some embodiments, the IAM enforces role-based access control, allowing authorized users to access specific datasets or functionalities within the clinical assessment system. In some embodiments, the compliance audit trail logs access attempts, modifications, and system interactions, ensuring traceability and accountability. It supports compliance with regulatory standards, including HIPAA and GDPR.

Within the data storage and management core 906, the patient health data is securely stored and managed. This data storage and management core 906 ensures that the patient health data is structured, indexed, and accessible for clinical analysis and AI/ML model training (e.g., the trained LLM 102). Additionally, ongoing security assessments are performed, which include vulnerability scans, penetration testing, and real-time monitoring to identify and mitigate emerging threats.

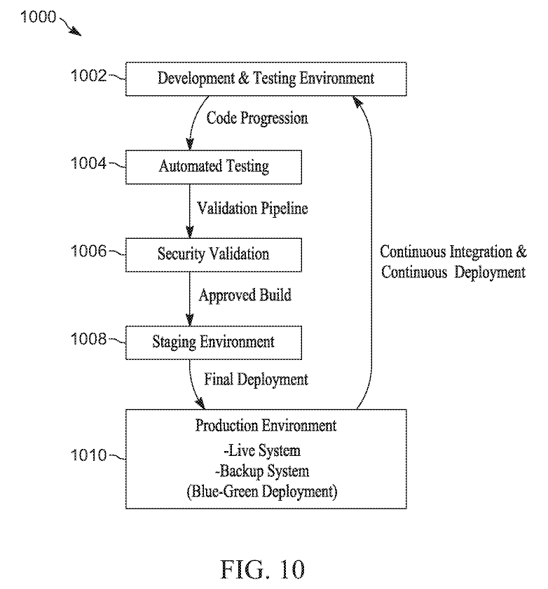

Referring now to FIG. 10, an exemplary flow diagram 1000 of testing, deployment, and updates of the clinical assessment system 100, is illustrated, in accordance with an embodiment of the present disclosure. In some embodiments, the flow diagram 1000 may be carried out by one or more processors/components of the clinical assessment system 100. In some embodiments, the flow diagram 1000 may be carried out by one or more processors external to system 100. This flow diagram 1000 depicts the systematic approach for continuous integration (CI) and continuous deployment (CD) of the clinical assessment system 100.

The initial code progression of the clinical assessment system 100 occurs in the development and testing environment 1002. This environment 1002 serves as the workspace for developing new features, fixing bugs, and implementing updates to the clinical assessment system 100. Once changes are made, the initial code enters the testing pipeline for further validation. The next phase involves automated testing 1004, which includes running a series of automated test cases designed to identify bugs, verify functionality, and ensure code quality. This step may include unit tests, integration tests, and regression tests. Automated testing ensures that new code does not disrupt existing system functionalities. Following successful automated testing, the code proceeds to the security validation phase 1006, where it undergoes rigorous checks to identify and mitigate potential vulnerabilities. This may include static and dynamic application security testing, penetration testing, and compliance verification with healthcare industry standards such as HIPAA. The code that passes all security checks may be approved for further deployment.

After security validation, the code enters the staging environment 1008, which mimics the production environment. This staging phase allows for live testing of the approved build in a controlled environment to identify any issues that might arise in a real-world setting. This block helps ensure that the system can handle expected user loads and provides a seamless user experience.

Upon successful validation in the staging environment, the system proceeds to the production environment 1010 for final deployment. The production environment employs a blue-green deployment strategy, where identical or substantially identical environments (e.g., live system and backup system) are maintained. During deployment, updates are first applied to the backup system (green environment) while the live system (blue environment) continues to operate without interruptions. Once the updates are validated in the backup system, traffic is rerouted to it, effectively making it the new live system. This approach minimizes downtime and ensures rollback capability in case of deployment issues.

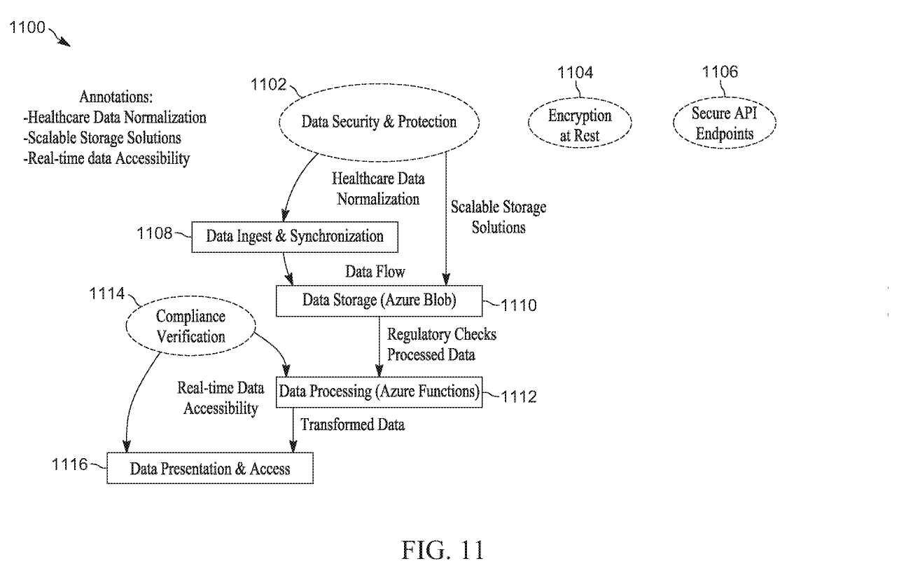

Referring now to FIG. 11, an exemplary architectural diagram 1100 of cloud-based data management, is illustrated, in accordance with an embodiment of the present disclosure. The architectural diagram 1100 depicts the multi-layered approach employed for the secure, scalable, and efficient management of healthcare data within the clinical assessment system 100.

The data management workflow includes data ingest and synchronization 1108, which is responsible for aggregating data from various input sources such as the wearable devices 138, the patient data repository 106 that includes patient-reported symptoms 140, input 142, and the EMR/EHR 144. Data security and protection 1102 ensures that patient health data operations adhere to stringent security protocols. This includes the implementation of encryption at rest 1104, which safeguards stored data using encryption technologies, and secure API endpoints 1106, which protect data during transmission to and from external systems. Once ingested, the data is routed to data storage 1110, which utilizes cloud storage solutions such as Azure Blob for scalable and secure storage. The storage architecture is optimized to handle large volumes of healthcare data efficiently while maintaining redundancy for data integrity. The next layer, data processing 1112, employs serverless computing capabilities such as Azure Functions to perform complex transformations and analyses on the raw data. This block ensures that the processed data meets the system's requirements for subsequent stages, such as clinical insights generation. The architecture incorporates a compliance verification 1114 module, which performs regulatory checks to ensure that all data operations are in compliance with healthcare standards and regulations such as HIPAA and GDPR. This module also audits the data handling processes to maintain accountability and transparency. The processed and verified data is then made available through the data presentation and access 1116 layer, which delivers transformed data to the system's various interfaces, including the patient interface 110 and clinician interface 112. This layer ensures real-time data accessibility and supports seamless integration with external systems.

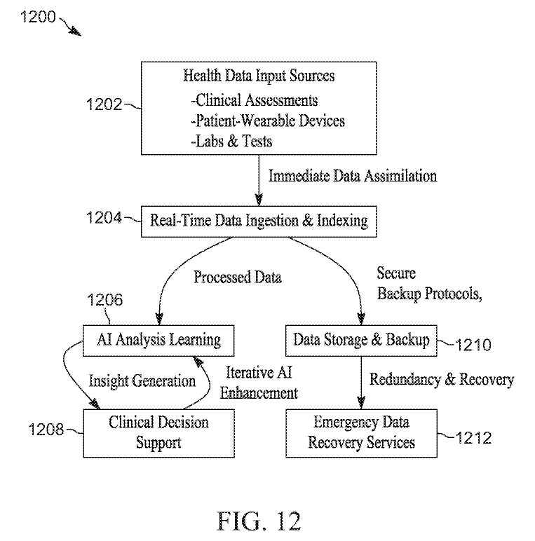

Referring now to FIG. 12, an exemplary flow diagram 1200 of real-time data handling within the clinical assessment system 100, is illustrated, in accordance with an embodiment of the present disclosure. In some embodiments, the flow diagram 1100 may be carried out by one or more processors/components of the clinical assessment system 100. In some embodiments, the flow diagram 1100 may be carried out by one or more processors external to system 100. The flow diagram 1200 demonstrates the systematic processing of patient health data. The process includes health data input sources 1202, which include the wearable devices 138 and the patient data repository 106 that includes patient-reported symptoms 140, input 142, and the EMR/EHR 144.

The patient health data is passed to the real-time data ingestion and indexing module 1204, which organizes and indexes the patient health data for efficient processing. This block ensures that the patient health data is accessible and ready for use by various system components. The indexed data is subsequently bifurcated into one or more processing streams, AI analysis and learning module 1206 and data storage and backup module 1210. The AI analysis and learning module 1206 processes the indexed data to generate clinical insights through iterative AI enhancement. This AI analysis and learning module 1206 includes capabilities for real-time updates and refinement of the trained LLM 102. The insights generated by this module 1206 are directed to the clinical decision support module 1208, which provides healthcare providers with actionable recommendations and diagnoses to optimize patient care. Simultaneously (or subsequently), the processed data is stored securely in the data storage & backup module 1210. This 1210 module ensures that the data is maintained with redundancy protocols. Additionally, this data storage and backup module 1210 enables secure backup protocols to protect the data and enable seamless recovery in case of system failures. The emergency data recovery services module 1212 works in conjunction with the data storage and backup systems. The emergency data recovery services module 1212 ensures that critical data can be retrieved quickly during emergencies, minimizing downtime and ensuring the continuity of patient care.

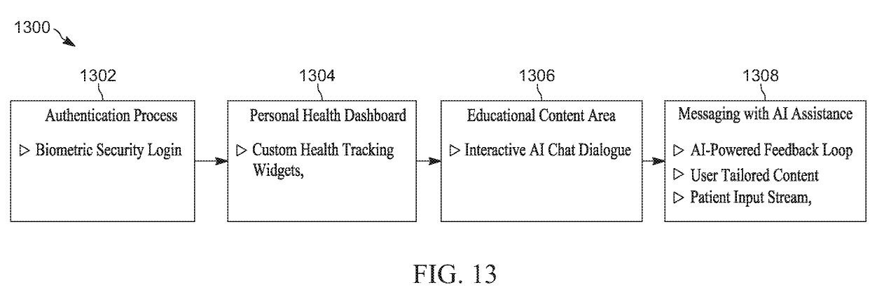

Referring now to FIG. 13, an exemplary schematic diagram 1300 of the patient interface 110, is illustrated, in accordance with an embodiment of the present disclosure. The patient interface 110 provides a user-centric platform designed to enable seamless interaction between patients and the healthcare system while offering personalized features to enhance patient engagement and care management. The process includes the authentication process 1302, which ensures secure access to the patient interface 110. The authentication process may involve a biometric security login, such as fingerprint scanning, facial recognition, or other biometric authentication methods. Upon successful authentication, the patient interface 110 transitions to the personal health dashboard 1304, which serves as the primary hub for the patient. The personal health dashboard 1304 includes custom health tracking widgets, allowing patients to monitor vital statistics, medication schedules, and other health parameters. These widgets can be tailored to the specific needs and preferences of individual patients, enhancing usability and personalization.

The patient interface 110 further incorporates an educational content area 1306, designed to empower patients with knowledge about their conditions and care plans. This area includes an interactive AI chat dialogue, where patients can ask questions, receive real-time answers, and engage with AI-powered educational resources. This feature promotes patient awareness and enables informed decision-making regarding their healthcare. The final component of the patient interface is messaging with AI assistance 1308, which enables seamless communication between patients and the system.

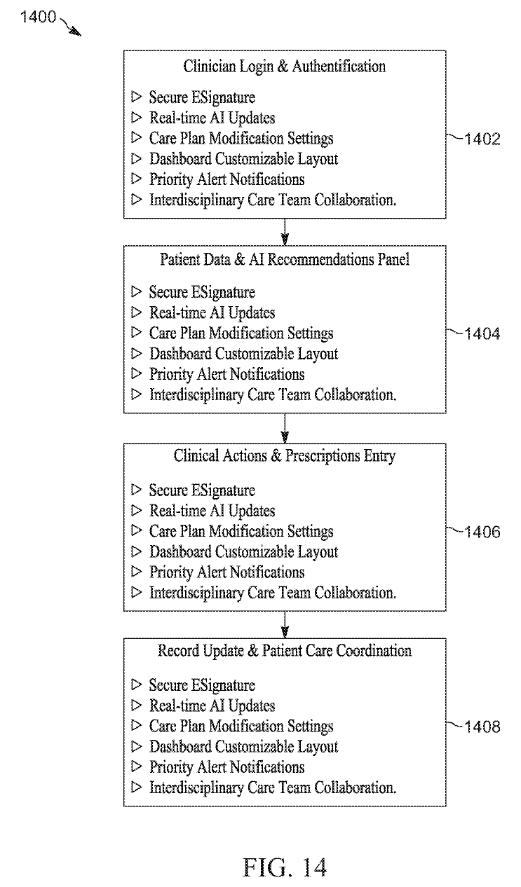

Referring now to FIG. 14, an exemplary schematic diagram 1400 of the clinician interface 112, is illustrated, in accordance with an embodiment of the present disclosure. The clinician interface 112 is designed to enable efficient clinical workflows, enabling healthcare providers to manage patient data, review AI-generated recommendations, and perform critical actions seamlessly.

The clinician interface 112 includes a clinician login and authentication module 1402, which ensures secure access to the clinical assessment system 100. The clinician login and authentication module 1402 includes features such as “Secure E-Signature” for authentication and authorization of the clinician, “Real-Time AI Updates” for keeping clinicians informed about patient statuses, and “Care Plan Modification Settings” for customizing treatment plans. Additionally, the module 1402 supports a “Dashboard Customizable Layout”, allowing clinicians to tailor the clinician interface 112 to their specific workflow needs. Features such as “Priority Alert Notifications” highlight critical cases requiring immediate attention, and “Interdisciplinary Care Team Collaboration” enables communication and coordination among different healthcare providers.

After logging in, clinicians access the patient health data and AI recommendations panel 1404, which consolidates patient information and AI-driven clinical insights. This panel includes tools for Secure E-Signature, enabling clinicians to approve AI-generated recommendations securely. Real-Time AI Updates provide instant visibility into evolving patient data, while care plan modification settings allow adjustments to treatment protocols. The clinical actions and prescriptions entry module 1406 enables clinicians to input treatment decisions, orders, and prescriptions. This clinical actions and prescriptions entry module 1406 also incorporates secure E-signature for validation, real-time AI updates for contextual guidance, and care plan modification settings for dynamic changes. The customizable dashboard layout enhances usability, and notifications and collaboration tools ensure cohesive care delivery across teams. The record update and patient care coordination module 1408 enables updating patient records and coordinating care activities. The record update and patient care coordination module 1408 integrates secure electronic signature functionality, ensuring compliance and security, along with real-time AI updates for keeping records current. Clinicians can utilize care plan modification settings and a dashboard customizable layout to manage patient data effectively. Priority alert notifications ensure timely interventions, while interdisciplinary care team collaboration supports patient care planning.

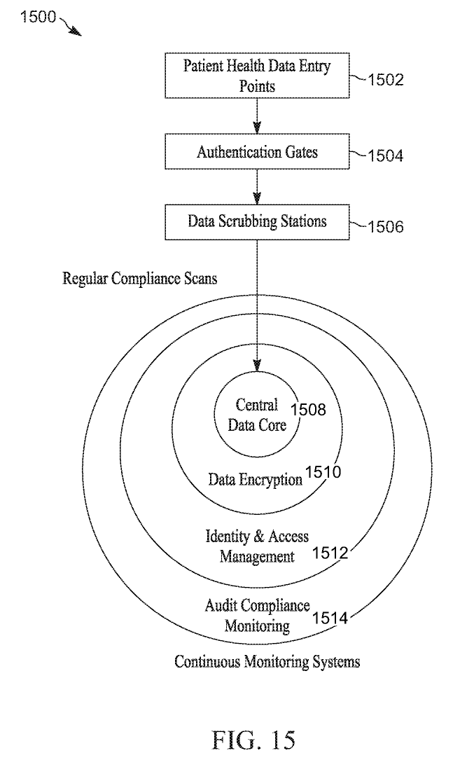

Referring now to FIG. 15, an exemplary flow diagram 1500 of data security and privacy protocol within the clinical assessment system 100, is illustrated, in accordance with an embodiment of the present disclosure. In some embodiments, the flow diagram 1500 may be carried out by one or more processors/components of the clinical assessment system 100. In some embodiments, the flow diagram 1500 may be carried out by one or more processors external to system 100. The protocol ensures comprehensive protection for sensitive data through a multi-layered approach, encompassing data entry, authentication, and core-level encryption with compliance monitoring.

The protocol includes user data entry points 1502, where patient health data is collected through designated interfaces such as the patient interface 110, the clinician interface 112, or input sources such as the wearable devices 138 and the patient data repository 106 that includes the patient-reported symptoms 140, input(s) 142, and EMR/EHR 144. The input sources may also include the historical medical data 146, and DSM data 148. These entry points are designed to handle various forms of input, including structured data, free text, and multimedia files. The patient health data collected at these points undergoes preliminary checks for validity and completeness. The collected patient health data proceeds to authentication gates 1504, which verify the credentials of patients accessing the clinical assessment system 100. This layer includes multifactor authentication (MFA), biometric verification, and/or secure token-based access to prevent unauthorized entry. The authentication gates ensure that verified personnel or systems can proceed further into the workflow, maintaining data integrity and restricting unauthorized access.

After authentication, the patient health data may be directed to data scrubbing stations 1506, where it is cleansed and normalized. These stations remove redundant, erroneous, or irrelevant information and apply data transformation techniques to ensure consistency and standardization. In some embodiments, the data scrubbing stations utilize NLP algorithms to extract meaningful entities/content from unstructured text and semantic indexing techniques to align the data with predefined schemas. Following data scrubbing, patient health data enters the central data core 1508, which acts as a repository for securely storing and processing data. The core employs advanced data encryption techniques, depicted as data encryption 1510, to ensure that stored data remains confidential and protected from unauthorized access. Encryption is performed using industry-standard protocols such as AES-256 to safeguard the information both at rest and during transit.

Surrounding the central data core is an identity and access management layer 1512, which governs permissions and access controls. This layer ensures that only authorized users and systems can retrieve or modify data. Role-based access controls (RBAC) and dynamic policy enforcement are implemented to restrict access based on user roles and the sensitivity of the data. An outer layer of the protocol includes audit compliance monitoring 1514, which conducts regular compliance scans and audits to ensure adherence to regulatory standards, such as HIPAA, GDPR, or FHIR guidelines. This layer enables real-time monitoring of system activities, generating logs for anomaly detection and forensic investigations in case of a breach. The framework may be supported by continuous monitoring systems, which ensure the protocol remains robust and adaptable to emerging threats. These systems utilize machine learning models to detect and mitigate potential vulnerabilities dynamically.

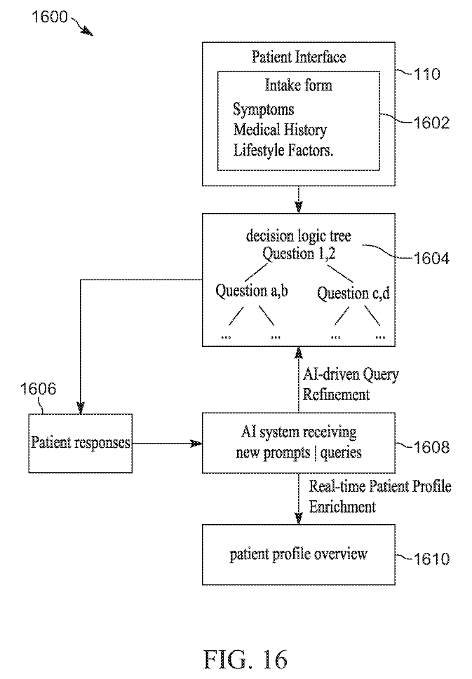

Referring now to FIG. 16, an exemplary flow diagram 1600 of interactive prompting and data capture process during patient intake, is illustrated, in accordance with an embodiment of the present disclosure. In some embodiments, the flow diagram 1600 may be carried out by one or more processors/components of the clinical assessment system 100. In some embodiments, the flow diagram 1600 may be carried out by one or more processors external to system 100. This process utilizes the patient interface 110 and advanced AI-driven mechanisms to collect, refine, and analyze patient data, ensuring a comprehensive and personalized intake experience.

The process begins at the patient interface 110, which includes an intake form 1602. The intake form 1602 captures various data points such as patient-reported symptoms, medical history, and lifestyle factors provided by the patient. The patient interface 110 may be presented in multiple formats, including web-based forms, mobile applications, or voice-enabled systems, offering flexibility in patient interaction. The captured patient health data is processed by a decision logic tree 1604, which operates as a branching algorithm to guide the interaction. The decision logic tree presents questions (e.g., "Question 1, 2") that branch into more specific queries ("Question a, b" and "Question c, d") depending on the patient’s input. This mechanism ensures that data collection is both comprehensive and relevant, dynamically tailoring the flow of questions to the patient’s specific condition or context.

Based on the received patient health data (and/or other data), patient responses 1606 are transmitted to an AI-driven system 1608 for refinement. The AI system 1608, receiving new prompts and queries, utilizes the trained LLM 102 to analyze patient responses and generate additional prompts as needed. This system 1608 employs advanced algorithms to refine the line of questioning to ensure that relevant information is captured in real time. This iterative refinement may include semantic analysis and contextual understanding of patient-provided data. The responses and refined prompts are used for real-time patient profile enrichment 1610. The AI system consolidates and processes the collected data to create a comprehensive patient profile. This profile includes detailed insights into the patient’s condition, potential risk factors, and personalized recommendations for subsequent clinical evaluation. The enriched profile is made accessible to healthcare providers using the clinician interface 112 for further review and decision-making.

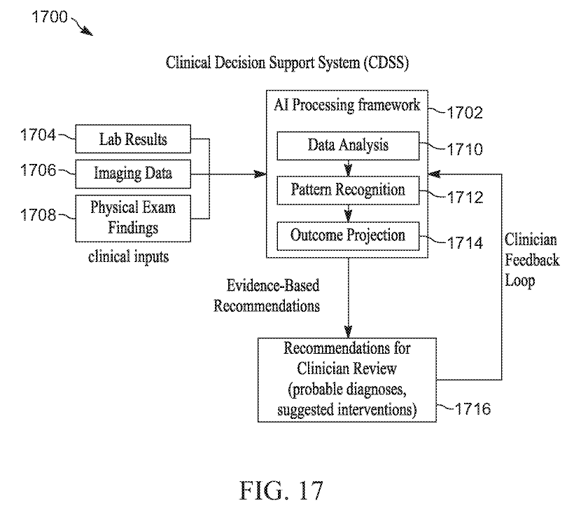

Referring now to FIG. 17, an exemplary flow diagram 1700 of analyzing clinical data and generating treatment recommendations within the clinical assessment system 100, is illustrated, in accordance with an embodiment of the present disclosure. In some embodiments, the flow diagram 1700 may be carried out by one or more processors/components of the clinical assessment system 100. For example, the clinical assessment system 100 may integrate multiple data sources, apply advanced AI-based processing, and provide actionable recommendations for clinician review. In some embodiments, the flow diagram 1700 may be carried out by one or more processors external to system 100.

The process includes the input of clinical data, which includes lab results 1704, imaging data 1706, and physical exam findings 1708. These clinical inputs represent patient data from various diagnostic and examination procedures. The system 100, for example, supports diverse input formats, including structured, semi-structured, and unstructured data, ensuring compatibility with various healthcare systems. The clinical inputs are processed by the AI processing framework 1702, which serves as the computational engine of the clinical assessment system 100. This framework includes one or more sub-module, for example data analysis 1710, pattern recognition 1712, and/or outcome projection 1714. The data analysis module 1710 preprocesses and contextualizes the clinical data, applying NLP, statistical methods, and semantic indexing to extract meaningful insights. The pattern recognition module 1712 utilizes machine learning algorithms to identify trends, anomalies, or correlations in the data, enabling the system to uncover hidden patterns that may inform clinical decision-making. The outcome projection module 1714 utilizes predictive analytics and probabilistic modeling to forecast potential outcomes based on the patient’s clinical profile, considering historical data, known treatment pathways, and relevant medical literature.

Once processed, the AI processing framework 1702 generates evidence-based recommendations that are forwarded to the recommendations module 1716 for clinician review. The module 1714 consolidates probable diagnoses, suggested interventions, and other relevant clinical actions into a structured output. The recommendations are presented to clinicians in an interpretable format through the clinician interface 112, enabling validation, modification, or acceptance. The clinical assessment system 100 includes a clinician feedback loop which allows healthcare providers to input their decisions and feedback into the system. This feedback is transmitted back to the AI processing framework 1702, where it is used to refine the pattern recognition and outcome projection processes, ensuring continuous improvement and learning over time.

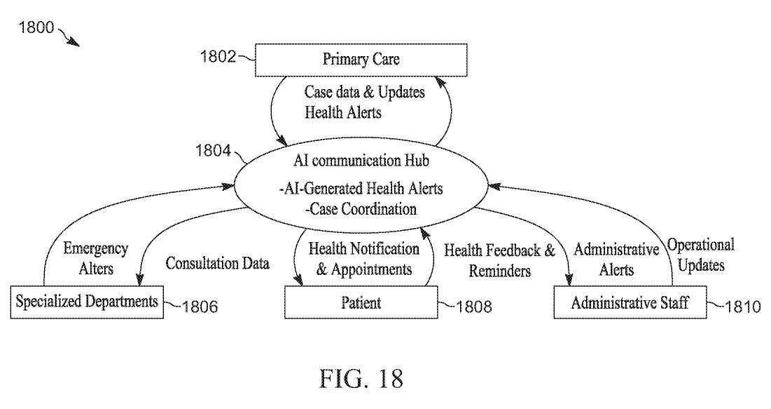

Referring now to FIG. 18, an exemplary flow diagram 1800 of communication flow and notification within the clinical assessment system, is disclosed, in accordance with an embodiment of the present disclosure. In some embodiments, the flow diagram 1800 may be carried out by one or more processors/components of the clinical assessment system 100. In some embodiments, the flow diagram 1800 may be carried out by one or more processors external to system 100.

The system enables real-time communication, health alerts, and/or coordination between various stakeholders, enhancing patient care and operational efficiency. The central component of the system is an AI communication hub 1804, which integrates various data sources and manages notifications, case updates, and health alerts. The AI communication hub 1804 utilizes advanced algorithms, which may include, but are not limited to, NLP algorithms for interpreting unstructured patient inputs, decision-tree algorithms for prioritizing notifications based on urgency, and machine learning algorithms for predictive analytics. These algorithms may work collaboratively to ensure seamless communication and efficient case coordination.

The communication flow originates from a primary care 1802, which provides case data, updates, and health alerts to the AI communication hub 1804. The AI communication hub 1804 may act as a primary point for initiating patient health monitoring and care coordination. Information from primary care is disseminated to relevant stakeholders through the hub. The communication flow proceeds to specialized departments 1806 which contribute specialized consultation data and receive emergency alerts from the AI communication hub 1804. This interaction ensures that critical patient cases are escalated and addressed promptly. Furthermore, patients 1808 receive health notifications, appointment reminders, and feedback through the AI communication hub 1804. Additionally, administrative staff 1810 interact with the system 100 by receiving operational updates and administrative alerts. The AI communication hub 1804 processes incoming data streams from all these entities, using AI algorithms (e.g., NLP algorithms for interpreting unstructured patient inputs, decision-tree algorithms for prioritizing notifications based on urgency, and machine learning algorithms for predictive analytics) to prioritize and route information efficiently. Health alerts and case updates are dynamically generated based on real-time patient conditions, operational changes, or new consultation inputs.

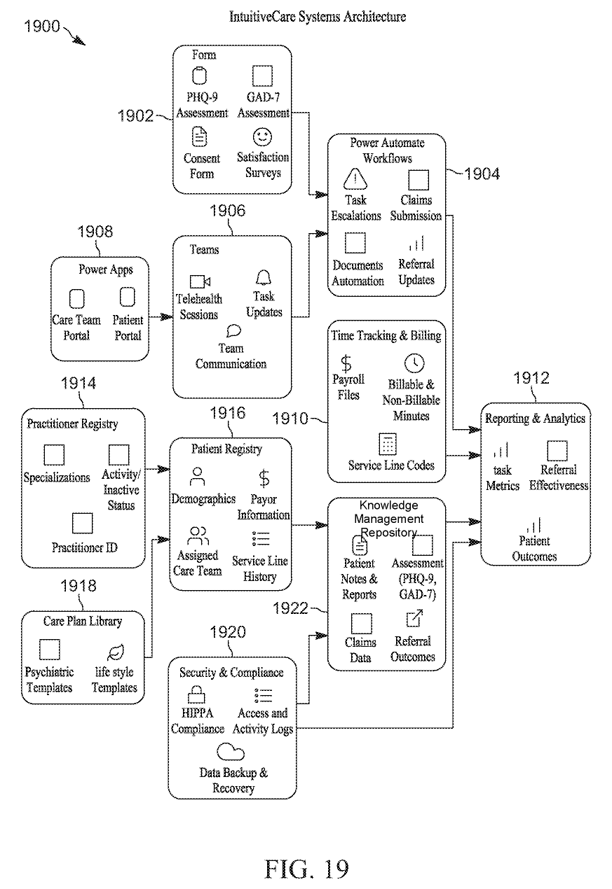

Referring now to FIG. 19, an exemplary architectural diagram 1900 depicting patient-centred care delivery, is illustrated, in accordance with an embodiment of the present disclosure. The architecture integrates various modules and workflows to enable efficient care coordination, secure data management, and actionable analytics, ensuring holistic patient care. The process includes form submissions 1902, which include assessments, consent forms, and surveys capturing patient data. These data flow into subsequent workflows for further processing and integration. Further, workflows and automation 1904 streamline tasks such as escalations, claims submissions, and document generation. Automated processes ensure timely notifications and updates across the system, enhancing operational efficiency. Further, communication modules 1906 enable real-time interactions among care teams. These modules support telehealth sessions, task updates, and secure messaging, enabling seamless collaboration. Further, portals 1908, including care team and patient interfaces, serve as access points for interacting with the system. The portals provide functionalities such as monitoring patient progress, scheduling tasks, and retrieving care plans. The time tracking and billing module 1910 captures billable and non-billable minutes associated with care delivery. The time tracking and billing module 1910 integrates with service line codes to support automated claims generation and financial reconciliation.

Additionally, reporting and analytics 1912 aggregate data from multiple modules to provide insights into task metrics, referral effectiveness, and patient outcomes. This module enables care teams and administrators to make data-driven decisions. The practitioner registry 1914 manages information about care providers, including their specializations, active or inactive statuses, and assigned roles. This registry dynamically links practitioners to relevant workflows. The care plan library 1918 provides pre-configured templates for psychiatric and lifestyle care. These templates can be customized in real-time by utilizing the patient health data (i.e., the biometric data, the historical medical data, and the patient-reported symptoms) and clinician inputs. For example, clinicians may use the clinician interface 112 to adjust the frequency of therapeutic sessions, update recommended lifestyle interventions, or add/remove milestones based on a patient’s progress. Real-time customization is facilitated by integration with the patient registry 1916, which ensures that up-to-date patient health data, such as changes in medical conditions or new diagnostic results, is automatically reflected in the care plan. The patient registry 1916 acts as the central repository for patient data, including demographics, payer information, assigned care teams, and service line history. This registry ensures a unified view of patient records, accessible across modules. The repositories integrate with workflows to automate document handling and reporting. Security and compliance module 1920 ensures adherence to regulatory requirements, such as HIPAA compliance. The Security and compliance module 1920 employs role-based access, data encryption, and backup mechanisms to safeguard sensitive information. The knowledge management repositories 1922 manage clinical notes, assessment reports, claims data, and referral outcomes.

The data flow and integration within this architecture enable seamless execution of key workflows to enhance patient care and operational efficiency. During patient onboarding, data collected is stored in the patient registry and linked to care teams, facilitating personalized care delivery. In the care plan development phase, templates from the care plan library are customized and shared with patients and clinicians for effective implementation. Service delivery is streamlined as care teams manage tasks and monitor progress using communication tools, while patients access real-time updates through dedicated portals. For claims and billing, time tracking data is utilized to generate claims, which are efficiently processed using automated workflows. Additionally, aggregated data may be generated and/or visualized through reporting and analytics, providing valuable insights into patient outcomes, team performance, and overall system efficiency.

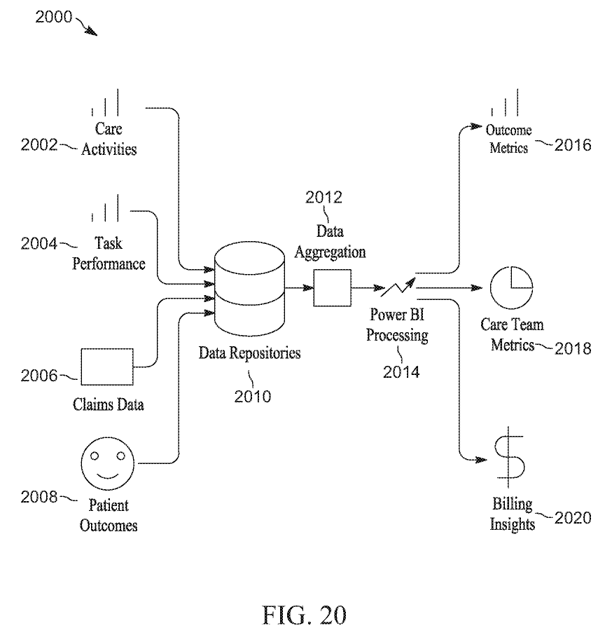

Referring now to FIG. 20, an exemplary data flow diagram 2000 of aggregation and processing of data from care activities, task performance, claims data, and patient outcomes into clinical insights using business intelligence dashboards, is illustrated, in accordance with an embodiment of the present disclosure. In some embodiments, the flow diagram 2000 may be carried out by one or more processors/components of the clinical assessment system 100. In some embodiments, the flow diagram 2000 may be carried out by one or more processors external to system 100.

At care activities 2002, data is collected from the patient registry and practitioner registry, including information related to care plans, clinical interventions, and overall healthcare delivery activities. At task performance 2004, escalations, task updates, and completion metrics are logged through automation tools such as Power Automate. These data reflect the efficiency and responsiveness of task management workflows. The claims data 2006 include data related to insurance claims, approvals, and denials, providing insight into financial workflows and reimbursement trends. At patient outcomes 2008, health outcomes such as recovery rates, adherence to treatment plans, and patient satisfaction metrics are tracked.

These data streams are stored in the data repositories 2010 (e.g., patient data repositories 106), which serve as centralized storage hubs for real-time updates from various system activities. The data repositories ensure secure and structured storage, maintaining the integrity and accessibility of logged information. The stored data undergoes data aggregation 2012, where it is combined and processed from multiple sources, such as patient records, task logs, and financial data, to generate a unified dataset. The aggregated data is then processed using business intelligence processing 2014, where advanced data visualization and analytics tools are applied to generate insights. These insights are categorized into one or more dashboards, for example outcome metrics 2016, care team metrics 2018, and/or billing insights 2020. The outcome metrics 2016 displays care effectiveness metrics, such as patient recovery rates, adherence to care plans, and health improvement statistics, providing clinicians with clinical insights into treatment success. The care team metrics 2018 highlights task completion rates, escalation resolution times, and team performance, enabling supervisors to monitor and optimize workforce efficiency. The billing insights 2020 provides a view of claims approval rates, revenue trends, and financial health, allowing administrative staff 1810 to manage financial workflows effectively.

Referring now to FIG. 21, an exemplary flow diagram 2100 for the patient health data processing within the clinical assessment system 100, is illustrated, in accordance with an embodiment of the present disclosure. In some embodiments, the flow diagram 2100 may be carried out by one or more processors/components of the clinical assessment system 100. In some embodiments, the flow diagram 2100 may be carried out by one or more processors external to system 100.

The flow diagram 2100 represents seamless integration of the patient health data into clinical insights and workflows for clinical decision-making. The process includes receiving assessment data 2104 (i.e., patient health data), related to a patient, through the patient interface 110. These data may include responses to clinical surveys, diagnostic assessments, or lifestyle-related inputs. Once submitted, the data is logged 2106 into a secure repository, ensuring the data is stored in the Patient Registry for subsequent analysis and reference. This secure logging ensures compliance with data protection standards and allows for real-time or retrospective analysis.

Following data logging, an automated workflow is triggered 2108 based on pre-configured thresholds or criteria within the system. These workflows are designed to detect critical scores such as high-risk PHQ-9 scores (e.g., ≥20, indicating severe depression), elevated GAD-7 scores (e.g., ≥15, reflecting severe anxiety), or abnormal biometric readings (e.g., heart rate variability below a certain threshold) in the assessment data. If critical scores are identified, the workflow enables escalation 2110 may ensure that urgent cases are flagged for immediate action. In parallel, the workflow notifies the assigned clinician 2112 to enable timely review and intervention tailored to needs of the patient.

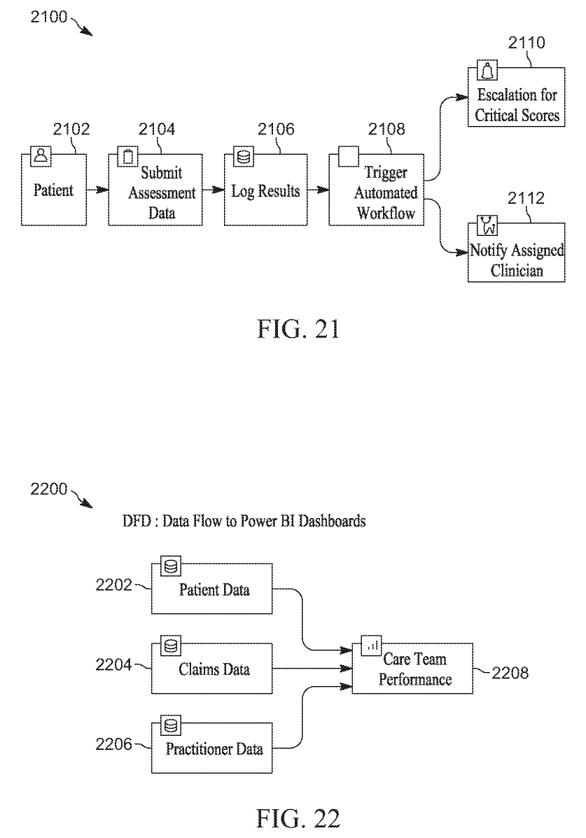

Referring now to FIG. 22, an exemplary data flow diagram 2200 depicting integration of various data streams into business intelligence dashboards for clinical insights, is illustrated, in accordance with an embodiment of the present disclosure. In some embodiments, the flow diagram 2200 may be carried out by one or more processors/components of the clinical assessment system 100. In some embodiments, the flow diagram 2200 may be carried out by one or more processors external to system 100.

The flow diagram 2200 highlights the aggregation of patient data 2202, claims data 2204, and practitioner data 2206 into a system to evaluate and enhance care team performance 2208. The process includes the patient data 2202, which includes demographic details, health records, and outcomes from clinical interactions. These data are continuously updated within the patient registry and securely integrated into the analytical framework. Claims data 2204 provides financial and administrative information, such as billing statuses, payment records, and claim approvals. These data ensure visibility into the revenue cycle and identifies opportunities for efficiency improvements. Practitioner data 2206 encompasses care team details, including task completion rates, specializations, and performance metrics, enabling a comprehensive understanding of resource utilization. These datasets are aggregated and fed into the care team performance module 2208, which powers real-time dashboards. The business intelligence dashboards serve as a unified platform for decision-making by visualizing key performance indicators such as patient outcomes, task efficiency, and financial metrics.

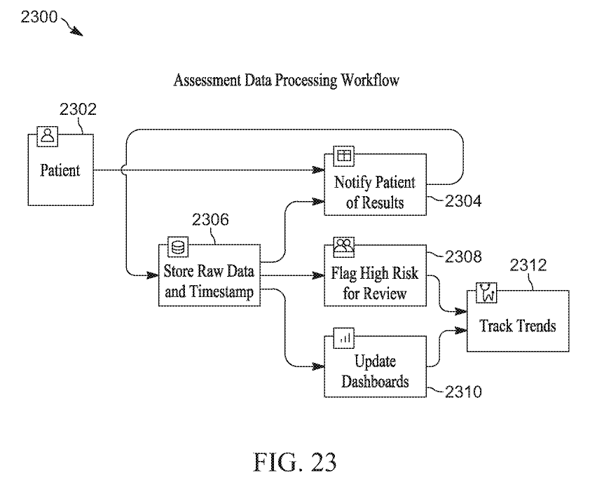

Referring now to FIG. 23, an exemplary workflow diagram 2300 for assessment data processing, showcasing the end-to-end handling of patient-submitted assessments to generate clinical insights, is illustrated, in accordance with an embodiment of the present disclosure. The workflow begins with a patient 2302 submitting an assessment, which may include standardized tools such as PHQ-9 or GAD-7, through a patient-facing interface, such as a web portal, mobile application, or clinician-guided submission. The assessment submission includes key metadata such as the assessment type, patient identification, and timestamp. This information forms the foundational layer for downstream processing. The submitted data is then forwarded to a storage module 2306, which securely stores the raw responses alongside the associated timestamp for traceability and compliance. This module acts as the repository for submitted assessments, ensuring data integrity and accessibility for subsequent workflows.

Upon successful submission, the system validates the assessment data to ensure required fields are populated and that responses match the expected format. For example, the system checks that numerical values are provided for all scored questions in a PHQ-9 form. If any data is incomplete or invalid, the workflow flags the submission as "incomplete" and sends a notification to the patient 2304 to resubmit the form, minimizing gaps in data processing. This validation step helps maintain the quality and accuracy of clinical insights generated later in the workflow. For example, the clinical insights may include, the severity of depressive symptoms based on PHQ-9 scores, correlations between patient-reported symptoms and historical medical data, prioritized care recommendations tailored to a patient’s risk level, personalized treatment plans that address comorbid conditions, and predictive health assessments to foresee potential complications or progression in the patient's condition.

Once validated, the system processes the data by calculating assessment scores based on predefined rules. For example, in the case of PHQ-9, the system sums the individual question scores to compute a total score. This calculated score, along with the raw responses, is stored within the assessment results table in the storage module 2306. The system then analyzes the calculated score to determine the patient’s risk level. For instance, a PHQ-9 score of 20 or above may be flagged as high risk, while scores in the range of 10-14 may be categorized as moderate risk. A score less than about 10 may be categorized as low risk. Based on the risk level, the system determines the appropriate course of action.

If the risk level is categorized as high or above a predefined threshold, the workflow triggers an automated flagging process 2308 to escalate the results to the assigned clinician or care team for urgent review. Notifications for flagged results are sent to the clinician through secure channels such as email, text messages, or integrated notifications in a clinician-facing dashboard. This ensures that high-priority cases receive immediate attention, minimizing the risk of delayed intervention. In parallel or sequentially, the system notifies the patient 2304 of their results along with any recommended next steps. For low-risk cases, the notification may include a reassurance message and educational resources to help the patient understand their assessment results. For moderate-risk cases, the notification may suggest scheduling a follow- up assessment or consultation. The system ensures that patient communication is clear, timely, and aligned with clinical guidelines.

The workflow further integrates advanced analytics by updating real-time dashboards 2310 with the assessment results. These dashboards provide a holistic view of trends across multiple patients, enabling clinicians and care administrators to monitor patterns such as an increase in high-risk assessments over time. The dashboards also allow for tracking individual patient progress, offering insights into the effectiveness of ongoing care plans. Additionally, the system includes a trend-tracking module 2312 that monitors historical data to identify long-term patterns and deviations. For example, a patient’s scores across multiple PHQ-9 assessments may be analyzed to determine whether their condition is improving or worsening. This module provides critical insights for clinicians to adjust treatment plans proactively.

The workflow incorporates error handling mechanisms to ensure reliability. If notifications to patients or clinicians fail due to technical issues, the system retries the notifications multiple times with predefined intervals. If repeated failures occur, the system escalates the issue to the system administrator for manual intervention. Similarly, if flagged assessments are not reviewed by clinicians within a predefined time frame, the system sends reminders and escalates the matter to supervisory staff to ensure timely action. To support compliance and auditing, the system logs actions and notifications in an audit trail, ensuring that steps of the workflow can be traced back for accountability. This may be used for meeting regulatory requirements such as HIPAA, which mandate secure handling of patient data.

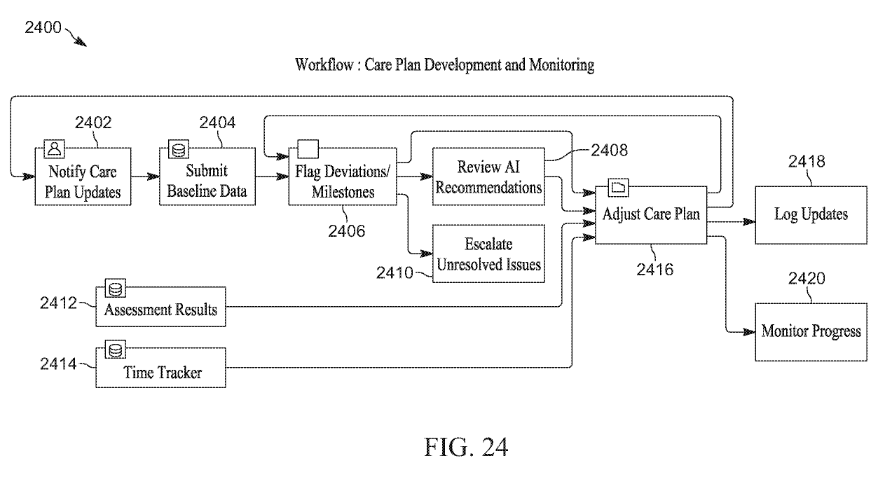

Referring now to FIG. 24, an exemplary workflow diagram 2400 for care plan development and monitoring (e.g., by monitoring system 126), depicting the processes for creating, updating, and tracking patient-specific care plans dynamically, is illustrated, in accordance with an embodiment of the present disclosure. This workflow integrates automated notifications, clinician oversight, and patient feedback, ensuring a responsive and adaptive approach to personalized care delivery.

The workflow begins with notifying the relevant care team or patient about updates or initiation of a care plan through a notification module 2402. Notifications may include a summary of the care plan objectives, milestones, and tasks, ensuring stakeholders are informed of the plan details. The patient or care team then submits baseline data 2404, including assessments, demographic details, and prior health records. These baseline data are critical for establishing the initial conditions of the care plan and serves as the foundation for subsequent monitoring and adjustments.

As the care plan progresses, the system monitors milestones and tasks, flagging any deviations or unmet goals through the milestone flagging module 2406. Deviations could include missed sessions, incomplete tasks, or a lack of measurable improvement in patient outcomes. These flagged issues are routed to the review module 2408, where AI-generated recommendations are presented to clinicians. The AI suggestions may include changes to session frequencies, introducing new interventions, or modifying outcome targets based on the identified deviations.

For unresolved or critical deviations, the system automatically triggers an escalation process 2410. This ensures that urgent issues, such as declining patient health metrics or repeated missed appointments, are brought to the attention of senior care managers or specialized clinicians. Escalations are communicated through secure notifications, ensuring timely intervention. Following a review of flagged deviations and AI recommendations, clinicians may adjust the care plan 2416 as necessary. Adjustments could include updating intervention strategies, revising milestones, or altering task deadlines to better align with the needs and/or progress of the patient. These updates are documented in the system using the log updates module 2418, ensuring that changes are recorded for compliance and future reference.

The adjusted care plan is monitored for adherence and effectiveness through the monitoring module 2420. This module integrates data from the assessment results table 2412 and the time tracker 2414 to provide a comprehensive view of patient progress. Key metrics such as adherence rates, assessment scores, and task completion timelines are analyzed to determine the effectiveness of the care plan. Real-time dashboards are updated to provide clinicians with clinical insights, enabling continuous monitoring and improvement of care delivery. The workflow includes automated notifications for patients and clinicians to ensure engagement and adherence. For example, patients may receive reminders for upcoming sessions or overdue tasks, while clinicians are alerted about flagged deviations or pending approvals for care plan adjustments. This ensures that parties are consistently aligned with the care plan objectives.

Error handling mechanisms are incorporated into the workflow to address potential issues such as missing data, unresponsive patients, or delays in clinician reviews. The system retries failed notifications and escalates unresolved issues to higher authorities, such as care managers, ensuring that no critical tasks are overlooked. Additionally, all actions and decisions within the workflow are logged in audit trails, providing transparency and supporting compliance with regulatory requirements.

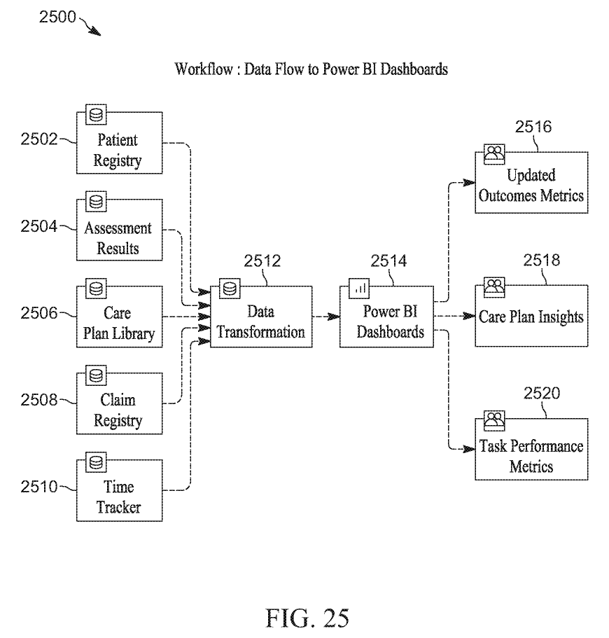

Referring now to FIG. 25, an exemplary workflow diagram 2500 for the seamless data flow into dashboards designed for real-time insights, is illustrated, in accordance with an embodiment of the present disclosure. This workflow consolidates clinical, operational, and administrative data from multiple sources to enable clinical insights into patient outcomes, clinician performance, task efficiency, and financial metrics, including claims processing. By centralizing and transforming data, the system ensures timely and accurate visualization for stakeholders, fostering data-driven decision-making.

The workflow begins with data consolidation from various sources. The patient registry 2502 serves as a repository for patient demographics, service line enrollments, and engagement history. Assessment results 2504 provide longitudinal trends and recent scores from assessments such as PHQ-9 or GAD-7, offering a quantitative measure of patient progress. The care plan library 2506 supplies active and completed care plan data, while the claims registry 2508 contributes financial and billing details, including CPT codes and claim statuses. The time tracker 2510 records task completion logs and the allocation of time for both billable and non-billable activities. These collected data form the foundation for the transformation process. Next, the data transformation module 2512 processes and cleanses the raw data to ensure compatibility with dashboard visualizations. The module maps database fields to pre-defined dashboard categories, such as mapping “PHQ-9 Score” to “Assessment Metrics.” Filters may be applied to focus on specific service lines, such as psychiatry, or aggregated timelines, such as weekly trends. This transformation step normalizes the data, preparing it for integration into the reporting system.

Once processed, the transformed data is sent to the dashboard engine 2514, where it is integrated into pre-configured templates. The dashboards automatically refresh to reflect the latest metrics, including patient trends, care plan adherence, task performance, and claims statuses. Updated outcomes metrics 2516 showcase patient recovery rates or trends in assessment scores, while care plan insights 2518 detail adherence rates and progress against established milestones. Task performance metrics 2520 visualize task completion rates, escalation trends, and time allocation efficiency. These dashboards serve as a single source of truth for monitoring and strategic planning. The system ensures proactive notifications to stakeholders about updates or critical changes in the dashboards. For example, care teams may receive alerts about flagged patient metrics, such as worsening assessment scores or overdue tasks, prompting immediate action. Notifications may also highlight operational metrics, such as an increase in claims rejection rates or task escalation rates exceeding predefined thresholds.

Error handling mechanisms are embedded within the workflow to ensure data accuracy and system reliability. The system logs errors during data retrieval, such as missing fields or failed queries, and attempts retries with incremental delays. If repeated attempts fail, a task is generated for manual reconciliation, and stakeholders are notified of persistent issues. Escalations are triggered for critical errors, such as discrepancies in claims data or prolonged dashboard synchronization failures, ensuring swift resolution. The decision-making framework within the workflow includes multiple escalation points. For example, if a patient registry lacks care plan updates or if discrepancies are found in task timestamps, the system alerts relevant stakeholders, such as clinicians or administrators, to resolve the issue. Similarly, flagged metrics from dashboards, such as overdue tasks or critical claims statuses, trigger notifications to ensure prompt action by the appropriate team.

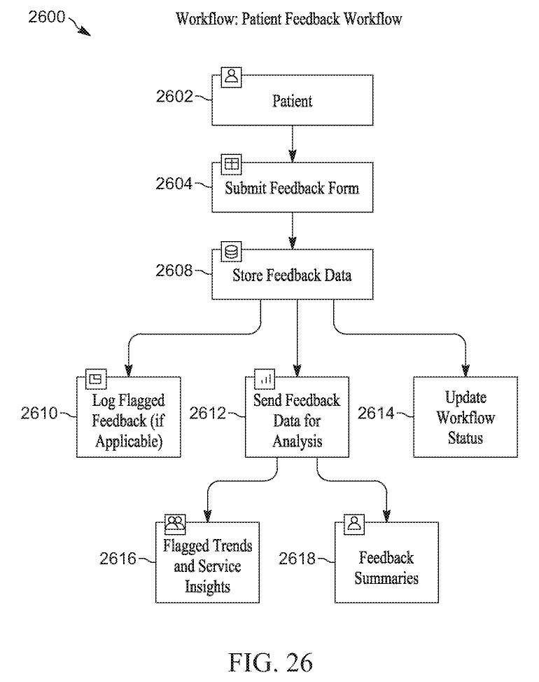

Referring now to FIG. 26, an exemplary workflow 2600 for collecting, processing, and analyzing patient feedback, is illustrated, in accordance with an embodiment of the present disclosure. This workflow ensures the efficient capture of patient experiences following critical interactions, such as therapy sessions or care plan reviews, and enables automated processing, escalation of flagged concerns, and continuous improvement of services.

The workflow including providing to a patient 2602 a feedback form using various channels, such as app notifications or email prompts. Feedback forms 2604 are designed to capture key metrics, including satisfaction ratings, open-ended comments, and specific session details. These forms may also include mandatory fields to ensure comprehensive data collection. Upon submission, the feedback data is securely stored 2608 in a feedback registry. This registry links the feedback to relevant patient records, interaction types (e.g., therapy sessions), and submission timestamps for traceability.

The stored feedback undergoes processing to identify clinical insights. In cases of flagged feedback, such as low ratings or negative comments, the system logs the flagged feedback 2610 for prioritized attention. Flagged responses are further categorized based on keywords indicating dissatisfaction or systemic issues, such as delays or unfulfilled expectations. Feedback data, including flagged and non-flagged entries, may also be transmitted 2612 for detailed analysis. The analysis phase generates aggregate trends, identifies recurring issues, and highlights specific areas for service improvement.

The system updates workflow status 2614 in real-time to reflect the completion of feedback collection, validation, and processing. This ensures end-to-end visibility into the feedback management lifecycle. Updated workflow statuses are logged to enable oversight and auditability of actions taken. The analyzed data contributes to flagged trends and service insights 2616, providing high-level visibility into systemic challenges or areas needing immediate intervention. Additionally, feedback summaries 2618 are compiled to offer individual clinicians or care teams concise insights into their interactions, promoting accountability and service enhancement.

The feedback workflow incorporates escalation logic for handling critical cases. For example, if a patient provides a satisfaction rating below a predefined threshold, such as 3 out of 5, the system automatically escalates the feedback to a care coordinator or supervisor. Similarly, flagged comments containing negative keywords trigger notifications to appropriate stakeholders for resolution. Unresolved escalations after a predefined time frame, such as 48 hours, are further escalated to departmental leads for immediate action. Error detection and fallback mechanisms ensure workflow reliability. If mandatory fields in feedback forms are incomplete, the system prompts the patient to resubmit. Errors during data storage, such as connectivity issues, trigger retries, and unresolved failures are logged for administrative review. Patients are notified of submission errors and provided with options to retry, ensuring a seamless experience.

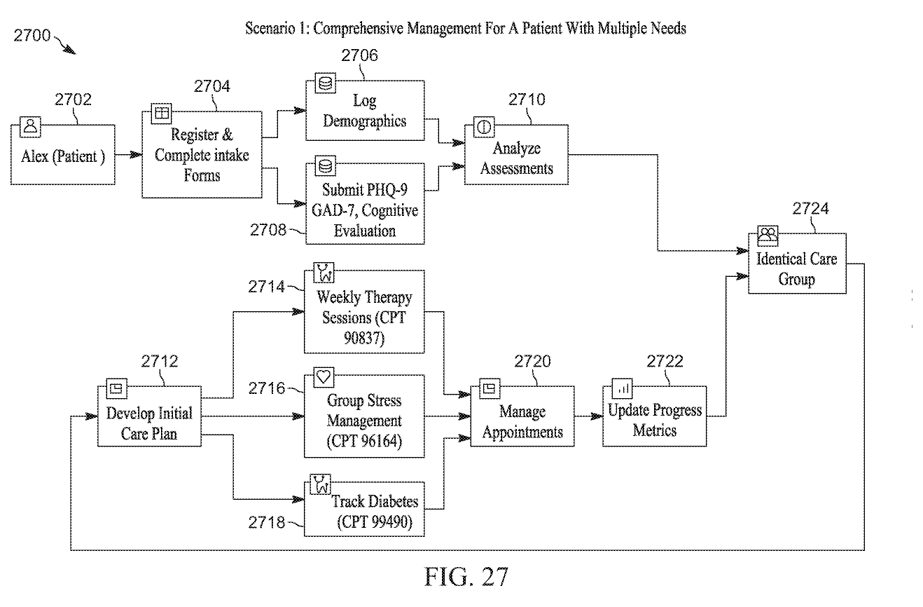

Referring now to FIG. 27, an exemplary use case scenario 2700 for the comprehensive management of a patient, is illustrated, in accordance with an exemplary embodiment of the present disclosure. Alex (i.e., a patient), who has multiple medical and mental health needs, including severe anxiety, Type 2 diabetes, and cognitive impairment. This workflow demonstrates the seamless integration of multiple service lines, AI-powered recommendations, and coordinated care to ensure holistic management of Alex’s conditions.

The workflow includes receiving completed intake forms 2704 from Alex 2702 through a patient-facing interface. These forms collect, for example, health history, demographics, and preliminary details, which are then logged into the log demographics system 2706. The process may include receiving a series of completed assessments 2708, including PHQ-9, GAD-7, and a cognitive evaluation. The assessment results, such as a high GAD-7 score of 19, are analyzed 2710, triggering notifications for necessary interventions, such as a psychiatric evaluation and enrolment in Chronic Care Management (CCM).

Based on the collected data and assessments, an initial care plan is created 2712. This care plan outlines specific interventions, such as weekly therapy sessions for anxiety 2714 (CPT 90837), group stress management sessions 2716 (CPT 96164), and diabetes monitoring and education 2718 (CPT 99490). The care plan is tailored to address Alex’s physical and mental health needs holistically, combining psychiatric services, lifestyle interventions, and chronic care management. Throughout the process, appointments are managed 2720 to ensure Alex has access to scheduled sessions and evaluations. Progress metrics are updated 2722 in real-time to monitor his outcomes across various parameters, such as anxiety improvement and diabetes control.

The system 100 utilizes these metrics to identify trends, such as plateauing improvements, and recommends adjustments to the care plan. For example, AI-powered analysis suggests increasing therapy frequency and incorporating family counselling to better address Alex’s anxiety. The workflow integrates Alex into an identical care group 2724 to provide peer support and shared experiences, which are beneficial for managing his stress and anxiety. Updates to his care plan and interventions are logged and monitored continuously to ensure that his treatment remains adaptive and responsive to his evolving needs.

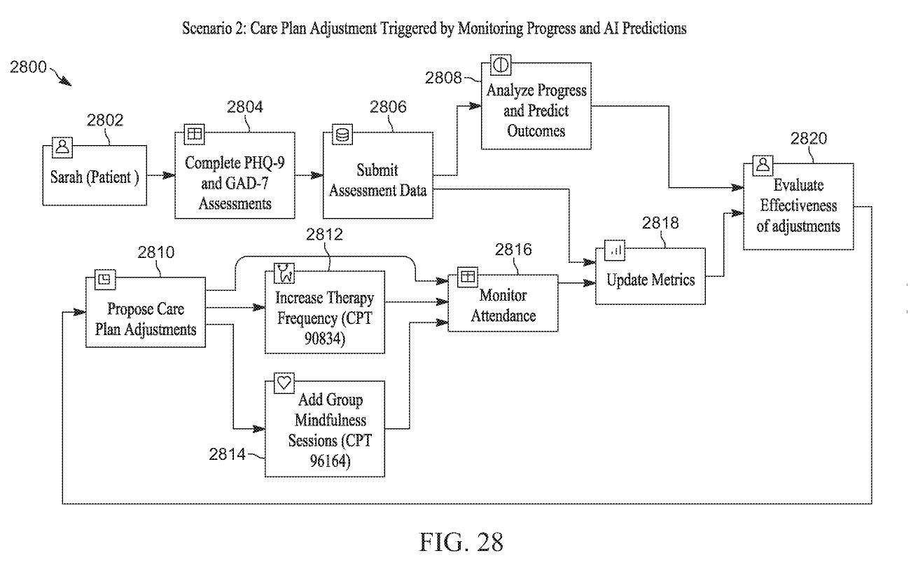

Referring now to FIG. 28, an exemplary scenario 2800, highlighting a care plan adjustment process triggered by monitoring patient progress and AI-based predictions, is illustrated, in accordance with an exemplary embodiment of the preset disclosure. This scenario focuses on Sarah, a patient whose progress stagnates, necessitating a reassessment and tailored modifications to her care plan to achieve better outcomes. The workflow integrates patient assessments, AI-generated recommendations, and care team interventions, ensuring seamless tracking and evaluation.

The process includes receiving completed PHQ-9 and GAD-7 assessments 2804 from Sarah 2802, which are integral to tracking her mental health progress. These assessments are submitted 2806 via a patient-facing interface, and the results are logged into the system. The data undergoes a thorough analysis 2808, where the system evaluates Sarah’s progress and predicts outcomes based on historical data, current metrics, and predictive analytics models. Based on the analysis, the system identifies the need for adjustments due to stagnation or lack of significant improvement. The care plan adjustment process 2810 is initiated, where the AI system (e.g., clinical assessment system 100) suggests actionable modifications, such as increasing the frequency of therapy sessions 2812 (CPT 90834) and incorporating group mindfulness sessions 2814 (CPT 96164). These adjustments are tailored to Sarah’s specific needs and historical patterns of responsiveness to interventions.

Once the proposed adjustments are approved, implementation is monitored 2816 to ensure Sarah attends the newly scheduled sessions and adheres to her updated care plan. Attendance and engagement data are logged and tracked in real time, providing the care team with immediate visibility into her compliance and progress. The system continuously updates metrics 2818 related to Sarah’s progress, which are visualized through dashboards for both care teams and administrators. This feedback loop enables timely evaluations of the effectiveness of the adjustments. If necessary, further modifications are made to the care plan to optimize outcomes. Finally, the effectiveness of the adjustments is thoroughly evaluated 2820. This involves comparing updated metrics with initial baselines to measure improvements and identify remaining challenges. The evaluation ensures that Sarah’s care remains dynamic and responsive to her evolving needs.

### METHODS

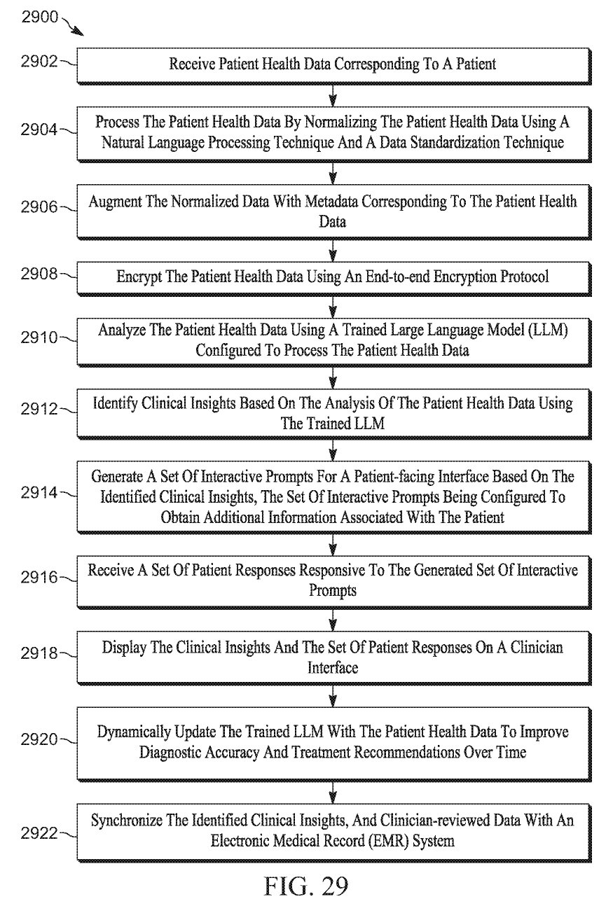

Referring now to FIG. 29, a flowchart of a method 2900 for generating a patient-specific clinical assessment, is illustrated, in accordance with an embodiment of the present disclosure. The method 2900 may additionally function to streamline data collection, analysis, and integration into care workflows. In some embodiments, the method 2900 functions to identify and prioritize patient-specific health concerns based on real-time data inputs, historical trends, and predictive analytics.

The method 2900 may be used for clinical applications such as mental health assessments, chronic disease management, and personalized treatment planning, but can additionally, or alternatively, be used for any suitable applications, clinical or otherwise. The method 2900 can be adapted to function for patient risk stratification, care plan customization, or any suitable decision-support system to improve the quality and efficiency of healthcare delivery.

At block 2902, the method 2900 may include receiving patient health data corresponding to a patient. In some embodiments, the patient health data may include one or more of biometric data, historical medical data, and patient-reported symptoms. For example, the patient interface 110 or input sources such as wearable devices 138 may perform this step. The patient interface 110 collects real-time inputs from patients, including manually reported symptoms and feedback submitted through mobile or web-based applications. In parallel, wearable devices 138, such as smartwatches or fitness trackers, provide continuous biometric monitoring, including heart rate, sleep patterns, and physical activity levels. Historical medical data may be sourced from EMR/EHR 144 or historical data repositories 146, which supply records of past diagnoses, lab results, and treatment plans. These data may also include psychiatric assessments, such as PHQ-9 or GAD-7 scores, sourced from patient data repositories 106.

Further at block 2904, the method 2900 may include processing the patient health data by normalizing the patient health data using a natural language processing technique and a data standardizing technique. The processing of the received patient health data further includes mapping the normalized data and the augmented data to a standardize medical coding system. For example, the trained LLM 102 (and/or external trained LLM 103) and the clinical workflow engine 108 perform this step in conjunction. The trained LLM 102 (and/or external trained LLM 103) may employ NLP to analyze unstructured patient-reported symptoms, clinician notes, and medical history. Using advanced NLP techniques, the trained LLM 102 (and/or external trained LLM 103) extracts relevant medical entities, such as diagnoses, symptoms, and treatment details, from free-text data sources. Concurrently (or subsequently), the clinical workflow engine 108, through its cognitive analysis module 116, applies data standardizing techniques to normalize diverse data formats. For example, numeric values from wearable devices 138 are transformed into standardized metrics (e.g., converting steps or activity levels into METs) while text data from EMR/EHR 144 is aligned to standardized terminologies.

Further at block 2906, the method 2900 may include augmenting the normalized data with metadata corresponding to the patient health data. In some embodiments, the metadata may be received from one or more input sources that may include electronic health records, wearable devices, or patient self-reports. For example, the clinical workflow engine 108 and the care management module 124 collectively perform this step. The clinical workflow engine 108, particularly through its insight generator 122, retrieves metadata from diverse input sources, including EMR/EHR 144, wearable devices 138, and patient-reported data from the patient interface 110. Metadata may include contextual details such as the timestamp of data capture, source reliability, measurement units, and environmental conditions affecting the data. For instance, heart rate readings from wearable devices 138 may be accompanied by metadata indicating the time of day, activity levels, or recent medication intake, which may be used for accurate interpretation. The care management module 124, utilizing its monitoring system 126, ensures that metadata is appropriately linked to the normalized patient health data. The metadata may include additional attributes, such as clinician notes from EMR/EHR 144, longitudinal trends from historical data repositories 146, and severity classifications derived from DSM data sources 148. For example, metadata for patient-reported symptoms 140 may include the frequency and intensity of symptoms over a specified period. Once the metadata is retrieved and contextualized, the context module 128 within the care management module 124 enriches the patient health data by associating it with relevant metadata, such as clinical context, demographic details, and past treatment responses. This enriched dataset provides a comprehensive view of the patient's condition, enabling advanced analytics and personalized recommendations by the trained LLM 102 (and/or external trained LLM 103).

Further at block 2908, the method 2900 may include encrypting the patient health data using an end-to-end encryption protocol. The security and compliance module 1922 is responsible for ensuring that patient health data is protected throughout its lifecycle. At this step, it employs end-to-end encryption protocols to safeguard sensitive information during transmission, storage, and processing. Encryption is applied to the normalized data and its associated metadata before it is transmitted between modules such as the patient interface 110, clinician interface 112, and input sources like EMR/EHR 144 or wearable devices 138.

Specifically, the module 1922 utilizes advanced encryption standards (AES) with key lengths of 256 bits, or equivalent cryptographic methods, to encrypt data at rest and in transit. For instance, data captured from wearable devices 138 is encrypted before being transmitted to the clinical workflow engine 108 for analysis. Similarly, encrypted channels, such as secure sockets layer (SSL) or transport layer security (TLS), are used for data exchange between the patient interface 110 and the clinician interface 112, ensuring compliance with regulatory requirements like HIPAA. To manage encryption keys securely, the security and compliance module 1922 employs role-based access controls and multi-factor authentication mechanisms, allowing authorized personnel or systems to decrypt and access the data. For example, a clinician accessing patient records through the clinician interface 112 would need to authenticate their identity, after which the decryption keys would be temporarily provided for viewing the data.

Further at block 2910, the method 2900 may include analyzing the patient health data using the trained LLM 102 (and/or external trained LLM 103) to process the patient health data. The analysis performed by the trained LLM may include contextually processing the patient health data to personalize the identified clinical insights using the trained LLM, the contextual processing being based at least in part on historical patient health data and real-time updates from at least one wearable device associated with the patient. For example, the trained LLM 102 (and/or external trained LLM 103) performs this step. The trained LLM 102 may be the analytical component of the clinical assessment system 100 and operates by utilizing its deep learning capabilities to analyse patient health data. The external trained LLM 103 may be an analytical component external to the clinical assessment system 100 that operates by using deep learning capabilities to analyze patient health and provide content, data, or recommendations to system 100. At this block, the trained LLM 102 (and/or external trained LLM 103) processes the encrypted, normalized, and augmented patient health data received from various sources, including patient-reported symptoms, historical electronic health records, and real-time data streams from wearable devices 138. During the analysis, the trained LLM contextually interprets the patient data using advanced NLP techniques. For instance, the LLM identifies patterns in historical patient data, such as recurring symptoms or prior treatment responses, and correlates them with real-time updates from wearable devices that provide metrics like heart rate, sleep quality, or physical activity levels. By incorporating metadata and real-time updates, the trained LLM 102 (and/or external trained LLM 103) enhances the depth and relevance of its analysis, ensuring that insights are both timely and personalized. The contextual processing performed by the trained LLM 102 (and/or external trained LLM 103) includes identifying potential health risks, recommending tailored interventions, and prioritizing clinical insights based on the patient's specific condition. For example, if wearable device data indicates elevated stress levels and historical data shows a history of anxiety, the LLM might recommend scheduling additional therapy sessions or mindfulness exercises. The contextual processing performed by the trained LLM 102 includes identifying potential health risks, recommending tailored interventions, and prioritizing clinical insights based on the patient's specific condition. For example, if wearable device data indicates elevated stress levels and historical data shows a history of anxiety, the LLM might recommend scheduling additional therapy sessions or mindfulness exercises.

Further at block 2912, the method 2900 may include identifying clinical insights based on the analysis of the patient health data using the trained LLM 102 (and/or external trained LLM 103). In some embodiments, the clinical insights may include potential diagnoses, treatment recommendations, and prioritized patient conditions. The trained LLM 102, as an analytical component of the clinical assessment system 100, is designed to derive meaningful and actionable clinical insights from the processed patient health data. After performing contextual analysis on the patient’s historical and real-time data, the LLM proceeds to identify specific clinical insights tailored to the patient's condition. To achieve this, the trained LLM 102 (and/or external trained LLM 103) employs RAG techniques, which allow it to dynamically retrieve relevant data from connected repositories such as the historical data repositories 146 and DSM data sources 148. The retrieved information is then combined with the current patient data to generate insights that are both comprehensive and individualized. The identification process relies on RAG techniques employed by the LLM, allowing it to dynamically pull relevant data from healthcare knowledge databases, including DSM data sources 148, historical data repositories 146, and medical literature. This ensures that the generated insights are up-to-date and aligned with current medical standards.

Further at block 2914, the method 2900 may include generating a set of interactive prompts for a patient-facing interface based on the identified clinical insights, the set of interactive prompts being configured to obtain additional information associated with the patient. The generation of the set of interactive prompts may further include dynamically adapting the set of interactive prompts, based on the set of patient responses, using a decision-tree algorithm implemented by the trained LLM 102 (and/or external trained LLM 103). The process of generating the interactive prompts begins with the trained LLM 102, which integrates the clinical insights identified in block 2912. The insights, such as potential diagnoses, treatment recommendations, or prioritized patient conditions, serve as the foundational input for crafting patient-specific prompts. These prompts are displayed through the patient interface 110, designed to collect additional contextual data and refine the system's understanding of the patient's condition.

Further at block 2916, the method 2900 may include receiving a set of patient responses responsive to the generated set of interactive prompts. The receipt of patient responses serves as a component of the clinical assessment workflow, enabling the system to gather contextual, real-time data directly from the patient. The patient interface 110, integrated with the clinical assessment system 100, enables this interaction by presenting the prompts generated in block 2914 and capturing the corresponding responses.

Further at block 2918, the method 2900 may include displaying the clinical insights and the set of patient responses on a clinician interface. The display of clinical insights and patient responses on the clinician interface 112 enables healthcare providers to make informed decisions based on comprehensive, real-time data. The clinician interface 112 is specifically designed to provide an intuitive and accessible platform for reviewing patient information, AI-generated insights, and patient-reported data.

Further at block 2920, the method 2900 may include dynamically updating the trained LLM with the patient health data to improve diagnostic accuracy and treatment recommendations over time. The dynamic update process involves integrating newly received patient health data and clinician feedback into the trained LLM 102 (and/or external trained LLM 103), enabling a refinement of LLM analysis and prediction models. This iterative learning process ensures that the system remains relevant and accurate in addressing patient needs.

Further at block 2922, the method 2900 may include synchronizing the identified clinical insights, and clinician-reviewed data with an EMR system. The step of synchronizing the identified clinical insights and clinician-reviewed data ensures seamless integration of patient-specific findings into the EMR/EHR 144, maintaining up-to-date and accessible medical records.

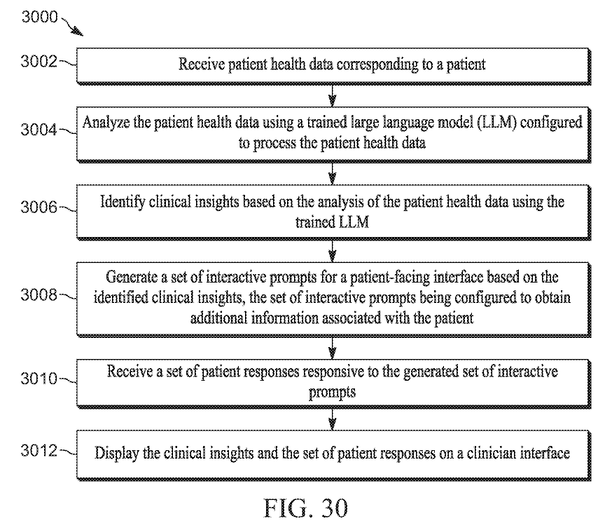

Referring now to FIG. 30, a flowchart of a method 3000 for generating a patient-specific clinical assessment, is illustrated, in accordance with an embodiment of the present disclosure. The method 3000 may additionally function to streamline data collection, analysis, and integration into care workflows. In some embodiments, the method 3000 functions to identify and prioritize patient-specific health concerns based on real-time data inputs, historical trends, and predictive analytics. The method 3000 may be used for clinical applications such as mental health assessments, chronic disease management, and personalized treatment planning, but can additionally, or alternatively be used for any suitable applications, clinical or otherwise. The method 3000 can be adapted to function for patient risk stratification, care plan customization, or any suitable decision-support system to improve the quality and efficiency of healthcare delivery.

At block 3002, the method 3000 may include receiving patient health data corresponding to a patient. In some embodiments, the patient health data may include one or more of biometric data, historical medical data, and patient-reported symptoms. For example, the patient interface 110 or input sources such as wearable devices 138 may perform this step. The patient interface 110 collects real-time inputs from patients, including manually reported symptoms and feedback submitted through mobile or web-based applications. In parallel, wearable devices 138, such as smartwatches or fitness trackers, provide continuous biometric monitoring, including heart rate, sleep patterns, and physical activity levels. Historical medical data may be sourced from EMR/EHR 144 or historical data repositories 146, which supply records of past diagnoses, lab results, and treatment plans. This data may also include psychiatric assessments, such as PHQ-9 or GAD-7 scores, sourced from patient data repositories 106.

At block 3004, the method 3000 may include analyzing the patient health data using a trained LLM to process the patient health data. The analysis performed by the trained LLM may include contextually process the patient health data to personalize the identified clinical insights using the trained LLM, the contextual processing being based at least in part on historical patient health data and real-time updates from at least one wearable device associated with the patient. For example, the trained LLM 102 (and/or external trained LLM 103) performs this step. The trained LLM 102 may be analytical component of the clinical assessment system 100 and operates by utilizing its deep learning capabilities to analyse patient health data. The external trained LLM 103 may be an analytical component external to the clinical assessment system 100 that operates by using deep learning capabilities to analyze patient health and provide content, data, or recommendations to system 100. At this block, the trained LLM 102 (and/or external trained LLM 103) processes the encrypted, normalized, and augmented patient health data received from various sources, including patient-reported symptoms, historical electronic health records, and real-time data streams from wearable devices 138. During the analysis, the trained LLM 102 (and/or external trained LLM 103) contextually interprets the patient data using advanced NLP techniques. For instance, the LLM identifies patterns in historical patient data, such as recurring symptoms or prior treatment responses, and correlates them with real-time updates from wearable devices that provide metrics like heart rate, sleep quality, or physical activity levels. By incorporating metadata and real-time updates, the trained LLM 102 (and/or external trained LLM 103) enhances the depth and relevance of its analysis, ensuring that insights are both timely and personalized. The contextual processing performed by the trained LLM 102 (and/or external trained LLM 103) includes identifying potential health risks, recommending tailored interventions, and prioritizing clinical insights based on the patient's specific condition. For example, if wearable device data indicates elevated stress levels and historical data shows a history of anxiety, the LLM might recommend scheduling additional therapy sessions or mindfulness exercises. The contextual processing performed by the trained LLM 102 (and/or external trained LLM 103) includes identifying potential health risks, recommending tailored interventions, and prioritizing clinical insights based on the patient's specific condition. For example, if wearable device data indicates elevated stress levels and historical data shows a history of anxiety, the LLM might recommend scheduling additional therapy sessions or mindfulness exercises.

Further at block 3006, the method 3000 may include identifying clinical insights based on the analysis of the patient health data using the trained LLM. In some embodiments, the clinical insights may include potential diagnoses, treatment recommendations, and prioritized patient conditions. The trained LLM 102, as an analytical component of the clinical assessment system 100, is designed to derive meaningful and actionable clinical insights from the processed patient health data. After performing contextual analysis on the patient’s historical and real-time data, the LLM proceeds to identify specific clinical insights tailored to the patient's condition. To achieve this, the trained LLM 102 employs RAG techniques, which allow it to dynamically retrieve relevant data from connected repositories such as the historical data repositories 146 and DSM data sources 148. The retrieved information is then combined with the current patient data to generate insights that are both comprehensive and individualized. The identification process relies on RAG techniques employed by the trained LLM 102, allowing it to dynamically pull relevant data from healthcare knowledge databases, including DSM data sources 148, historical data repositories 146, and medical literature. This ensures that the generated insights are up-to-date and aligned with current medical standards.

Further at block 3008, the method 3000 may include generating a set of interactive prompts for a patient-facing interface based on the identified clinical insights. The set of interactive prompts may be configured to obtain additional information associated with the patient. The generation of the set of interactive prompts may further include dynamically adapting the set of interactive prompts, based on the set of patient responses, using a decision-tree algorithm implemented by the trained LLM 102 (and/or external trained LLM 103). The process of generating the interactive prompts begins with the trained LLM 102 (and/or external trained LLM 103), which integrates the clinical insights identified in block 3006. The clinical insights, such as potential diagnoses, treatment recommendations, or prioritized patient conditions, serve as the foundational input for crafting patient-specific prompts. These prompts are displayed through the patient interface 110, designed to collect additional contextual data and refine the system's understanding of the patient's condition.

Further at block 3010, the method 3000 may include receiving a set of patient responses responsive to the generated set of interactive prompts. The receipt of patient responses serves as a component of the clinical assessment workflow, enabling the system to gather contextual, real-time data directly from the patient. The patient interface 110, integrated with the clinical assessment system 100, enables this interaction by presenting the prompts generated in block 3008 and capturing the corresponding responses.

Further at block 3012, the method 3000 may include displaying the clinical insights and the set of patient responses on a clinician interface. The display of clinical insights and patient responses on the clinician interface 112 enables healthcare providers to make informed decisions based on comprehensive, real-time data. The clinician interface 112 is specifically designed to provide an intuitive and accessible platform for reviewing patient information, the clinical insights, and the patient-reported symptoms.

In some aspects, the techniques described herein relate to a computer-implemented method that is HIPPA compliant. In general, the systems described herein may generate requisite warnings to protect the information contained in data accessed and/or stored by such systems in compliance with the Health Insurance Portability and Accountability Act (HIPPA). Accordingly, the systems and methods described herein provide compliance with HIPPA, and other privacy requirements regarding patient medical data, or other personal information. For example, the processes encrypting patient health data (e.g., using end-to-end encryption protocols as described at block 2908) are executed in such a fashion to be HIPPA compliant and requisite warnings are provided to protect the information contained in system 100 in compliance with HIPPA.

The systems and methods of the embodiments described herein and variations thereof can be embodied and/or implemented at least in part as a machine configured to receive a computer-readable medium storing computer-readable instructions. The instructions may be executed by computer-executable components integrated with the system and one or more portions of the processor 132 on the clinical assessment system 100 and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component may be a general or application-specific processor, but any suitable dedicated hardware or hardware/firmware combination can alternatively or additionally execute the instructions.

For instance, the processors 132, as described in FIG. 1B, may include specialized accelerators for machine learning tasks, such as GPUs or TPUs, to execute computationally intensive operations like training and inference for the trained LLM 102. These processors may retrieve the instructions from the memory 130, where the instructions are stored in non-transitory storage media, and execute tasks such as normalizing patient data, performing contextual analysis, and generating clinical insights. In some embodiments, the memory 130 may be communicably coupled to one or more of the processors 132. The memory 130 may store processor-executable instructions, which when executed by the one or more processors 132, cause the processor to carry out one or more instructions, as described throughout this disclosure in at least the flow diagrams herein.

Additionally, the instructions may define the workflows of various modules such as the clinical workflow engine 108, care management module 124, and security and compliance module 1922, ensuring that each component performs its designated functions in an integrated manner. The computer-readable medium may also include software libraries and frameworks enabling the system to interface with external devices, such as wearable devices 138, patient-facing interfaces 110, or clinician-facing interfaces 112.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used in the description and claims, the singular form “a”, “an” and “the” include both singular and plural references unless the context clearly dictates otherwise. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by ( + ) or ( - ) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A computer-implemented method for generating a patient-specific clinical assessment, the method comprising:
receiving, by a processor, health data corresponding to a patient, wherein the health data comprises one or more of: biometric data, historical medical data, and patient-reported symptoms;
analyzing, by the processor, the health data using a trained large language model (LLM), wherein the analyzing comprises contextually processing the health data based at least in part on the historical medical data corresponding to the patient, to generate a set of clinical insights comprising potential diagnoses, treatment recommendations, and prioritized patient conditions;
generating, by the processor, a set of interactive prompts for a patient interface, wherein the set of interactive prompts is generated based on the set of clinical insights and configured to obtain additional information associated with at least one of the potential diagnoses, treatment recommendations, and prioritized patient conditions;
dynamically adapting, by the processor, the set of interactive prompts based on a set of patient responses using a decision-tree algorithm implemented by the trained LLM, wherein the dynamically adapting comprises modifying subsequent prompts based on analysis of previous patient responses to obtain context-specific clinical information;
receiving, by the processor, the set of patient responses responsive to the dynamically adapted set of interactive prompts;
updating, by the processor, the set of clinical insights by processing the set of patient responses using the trained LLM, wherein the updating comprises modifying at least one of the potential diagnoses, treatment recommendations, or prioritized patient conditions based on the set of patient responses, to generate an enriched patient clinical profile; and
causing display of, by the processor, the enriched patient clinical profile on a clinician interface, the enriched patient clinical profile comprising the updated clinical insights and the set of patient responses.

2. The computer-implemented method of claim 1, further comprising:
processing, by the processor, the health data by normalizing the health data using a natural language processing technique and a data standardization technique; and
augmenting, by the processor, the normalized data with metadata corresponding to the health data,
wherein the metadata is received from one or more input sources comprising: electronic health records, wearable devices, or patient self-reports, and
wherein the metadata comprises temporal attributes, locational attributes, and categorical attributes corresponding to the health data.

3. The computer-implemented method of claim 2, wherein the processing of the received health data further comprises:
mapping, by the processor, the normalized data and the augmented data to a standardized medical coding system.

4. The computer-implemented method of claim 1, further comprising:
dynamically updating, by the processor, the trained LLM with the health data to improve diagnostic accuracy and treatment recommendations over time; and
synchronizing, by the processor, the set of clinical insights, and clinician-reviewed data with an electronic medical record (EMR) system.

5. The computer-implemented method of claim 1, wherein the analysis further comprises:
contextually processing the health data to personalize the set of clinical insights using the trained LLM, the contextual processing being based at least in part on historical health data and real-time updates from at least one wearable device associated with the patient.

6. The computer-implemented method of claim 1, further comprising:
enforcing role-based access controls for decryption permissions.

7. The computer-implemented method of claim 1, further comprising:
augmenting, by the processor, the trained LLM using a retrieval-augmented generation (RAG) technique,
wherein the RAG technique retrieves relevant data from a healthcare knowledge database comprising psychiatric research, historical patient data, treatment outcomes, and medical literature.

8. The computer-implemented method of claim 1, further comprising:
encrypting, by the processor, the health data using an end-to-end encryption protocol, wherein the encrypting comprises: encrypting patient responses received from a patient interface prior to processing by a trained large language model (LLM) and processing, by the processor, the health data within a security boundary.

9. The computer-implemented method of claim 1, further comprising:
evaluating, by the processor, at least one of the updated clinical insights against a predetermined clinical threshold; and
in response to the at least one updated clinical insight satisfying the predetermined clinical threshold, automatically generating a clinical notification directed to a designated care team member.

10. A computer-implemented system for generating a patient-specific clinical assessment, comprising:
a processor; and
a memory communicably coupled to the processor, wherein the memory stores processor-executable instructions, which when executed by the processor, cause the processor to:
receive health data corresponding to a patient, wherein the health data comprises one or more of: biometric data, historical medical data, and patient-reported symptoms;
analyze the health data using a trained large language model (LLM), wherein the analyzing comprises contextually processing the health data based at least in part on the historical medical data corresponding to the patient, to generate a set of clinical insights comprising potential diagnoses, treatment recommendations, and prioritized patient conditions;
generate a set of interactive prompts for a patient interface, wherein the set of interactive prompts is generated based on the set of clinical insights and configured to obtain additional information associated with at least one of the potential diagnoses, treatment recommendations, and prioritized patient conditions;
dynamically adapt the set of interactive prompts based on a set of patient responses using a decision-tree algorithm implemented by the trained LLM, wherein the dynamically adapting comprises modifying subsequent prompts based on analysis of previous patient responses to obtain context-specific clinical information;
receive the set of patient responses responsive to the dynamically adapted set of interactive prompts;
update the set of clinical insights by processing the set of patient responses using the trained LLM, wherein the updating comprises modifying at least one of the potential diagnoses, treatment recommendations, or prioritized patient conditions based on the set of patient responses, to generate an enriched patient clinical profile; and
cause display of the enriched patient clinical profile on a clinician interface, the enriched patient clinical profile comprising the updated clinical insights and the set of patient responses.

11. The computer-implemented system of claim 10, wherein the processor-executable instructions further cause the processor to:
process the health data by normalizing the health data using a natural language processing technique and a data standardization technique; and
augment the normalized data with metadata corresponding to the health data,
wherein the metadata is received from one or more input sources comprising: electronic health records, wearable devices, or patient self-reports, and
wherein the metadata comprises temporal attributes, locational attributes, and categorical attributes corresponding to the health data.

12. The computer-implemented system of claim 11, wherein to process the received health data, the processor-executable instructions cause the processor to:
map the normalized data and the augmented data to a standardized medical coding system.

13. The computer-implemented system of claim 10, wherein the processor-executable instructions further cause the processor to:
dynamically update the trained LLM with the health data to improve diagnostic accuracy and treatment recommendations over time; and
synchronize the set of clinical insights, and clinician-reviewed data with an electronic medical record (EMR) system.

14. The computer-implemented system of claim 10, wherein analyzing the health data comprises:
contextually processing the health data to personalize the set of clinical insights, the contextual processing being based at least in part on historical health data and real-time updates from at least one wearable device associated with the patient.

15. The computer-implemented system of claim 10, wherein the processor-executable instructions cause the processor to:
enforce role-based access controls for decryption permissions.

16. The computer-implemented system of claim 10, wherein the processor-executable instructions further cause the processor to:
augment the trained LLM using a retrieval-augmented generation (RAG) technique, wherein the RAG technique retrieves relevant data from a healthcare knowledge database comprising psychiatric research, historical patient data, treatment outcomes, and medical literature.

17. A non-transitory computer-readable storage medium for use in conjunction with a computing system, the computer-readable storage medium being configured to store executable program instructions that, when executed by the computing system, cause the computing system to perform operations comprising:
receiving patient health data corresponding to a patient, wherein the patient health data comprises one or more of: biometric data, historical medical data, and patient-reported symptoms;
analyzing the health data using a trained large language model (LLM), wherein the analyzing comprises contextually processing the health data based at least in part on the historical medical data corresponding to the patient, to generate a set of clinical insights comprising potential diagnoses, treatment recommendations, and prioritized patient conditions;
generating a set of interactive prompts for a patient interface, wherein the set of interactive prompts is generated based on the set of clinical insights and configured to obtain additional information associated with at least one of the potential diagnoses, treatment recommendations, and prioritized patient conditions;
dynamically adapting the set of interactive prompts based on a set of patient responses using a decision-tree algorithm implemented by the trained LLM, wherein the dynamically adapting comprises modifying subsequent prompts based on analysis of previous patient responses to obtain context-specific clinical information;
receiving the set of patient responses responsive to the dynamically adapted set of interactive prompts;
updating the set of clinical insights by processing the set of patient responses using the trained LLM, wherein the updating comprises modifying at least one of the potential diagnoses, treatment recommendations, or prioritized patient conditions based on the set of patient responses, to generate an enriched patient clinical profile; and
causing display of the enriched patient clinical profile on a clinician interface, the enriched patient clinical profile comprising the updated clinical insights and the set of patient responses.

18. The non-transitory computer-readable storage medium of claim 17, wherein the stored executable program instructions that, when executed by the computing system, further cause the computing system to perform operations comprising:
processing the patient health data by normalizing the patient health data using a natural language processing technique and a data standardization technique; and
augmenting the normalized data with metadata corresponding to the patient health data,
wherein the metadata is received from one or more input sources comprising: electronic health records, wearable devices, or patient self-reports, and
wherein the metadata comprises temporal attributes, locational attributes, and categorical attributes corresponding to the patient health data.

19. The non-transitory computer-readable storage medium of claim 18, wherein to process the received patient health data, the stored executable program instructions that, when executed by the computing system, further cause the computing system to perform operations comprising:
mapping the normalized data and the augmented data to a standardized medical coding system.

20. The non-transitory computer-readable storage medium of claim 17, wherein the stored executable program instructions that, when executed by the computing system, further cause the computing system to perform operations comprising:
dynamically updating the trained LLM with the patient health data to improve diagnostic accuracy and treatment recommendations over time; and
synchronizing the set of clinical insights, and clinician-reviewed data with an electronic medical record (EMR) system.
